(19) United States
(12) Patent Application Publication
(10) Pub. No.: US 2020/0140850 A1
(43) Pub. Date: May 7, 2020

(54) METHODS FOR ISOLATION AND QUANTIFICATION OF SHORT NUCLEIC ACID MOLECULES

(71) Applicant: TECHNION RESEARCH & DEVELOPMENT FOUNDATION LIMITED, Haifa (IL)

(72) Inventors: Ayelet LAMM, Zihron Ya'akov (IL); Alla FISHMAN, Haifa (IL)

(21) Appl. No.: 16/315,692

(22) PCT Filed: Jul. 6, 2017
(86) PCT No.: PCT/IL2017/050761

§ 371 Date: Jan. 7, 2019

Related U.S. Application Data
(60) Provisional application No. 62/359,245, filed on Jul. 7, 2016.

(51) Int. Cl. C12N 15/10; C40B 50/06; C40B 20/04

(52) U.S. Cl. CPC C12N 15/1006; C40B 20/04; C40B 50/06

(57) ABSTRACT

Methods, kits and compositions for separation, identification, and isolation of short nucleic acids (i.e., less than 100 nucleotides) of different length are provided. The invention further provides methods for preparation of small RNA libraries.

### FIELD OF INVENTION

The present invention is directed to kits and methods in the field of small nucleic acid isolation and quantification.

### BACKGROUND OF THE INVENTION

Small RNAs (sRNAs) are RNA molecules that play an important role in regulation of gene expression. One of the most important types of small RNAs is the microRNA (miRNA). MicroRNAs are conserved and function in RNA silencing as well as post-transcriptional regulation of gene expression. There are more than 1000 currently known microRNAs in humans, some of which are known to be associated with various diseases. In order to understand different cellular processes, the repertoire of cellular sRNAs and the abundance of each one of them must be revealed. One of the common methods for identifying and quantifying sRNAs in cells is to sequence the sRNAs by using high-throughput sequencing (HTS) platforms. For this purpose, a sRNA library is prepared by extracting sRNAs from cells followed by ligating oligonucleotide sequences to each end of the sRNA. These oligonucleotides, usually called “linkers” or “adaptors”, allow alignment of primers required for the subsequent processes of reverse transcription of sRNAs and their amplification. A reverse transcription step typically follows using a reverse transcriptase to produce cDNA molecules. The obtained library is next amplified by PCR and subjected to HTS. Subsequently the sequence data is analyzed to obtain the abundance of each of the sRNAs in the samples.

Preparing a library for HTS typically includes several steps, each dependent on different enzymatic reaction. Following each step, the desired reaction product is typically separated or cleaned off from the other reaction components and/or undesired products, to ensure efficiency of the subsequent steps. Products of ligation between adaptors may occur during preparation of sRNA libraries for HTS. This undesired by-product, commonly known as an “adaptor-dimer”, is generated when a 5′ adaptor is ligated directly to a 3′ adaptor. Thus, the cDNA that is generated by the reverse transcription reaction contains both the intended sRNA library products as well as the undesired adaptor-adaptor by-product. If the generation of the adaptor-adaptor by-product is not minimized or the generated by-product not removed, much of the PCR amplification components may be preferentially utilized to amplify the by-product instead of the library inserts of interest. Thus, sequencing capacity and reagents will be spent on sequencing this by-product, thereby limiting the yield of the RNA segment of interest. In order to minimize the production of the undesired adaptor-adaptor by-product, ligation of the two adaptors is performed sequentially and a purification step for removal of undesired by-products may be needed after each ligation of adaptor to sRNA.

Methods for isolating target nucleic acid molecules (e.g., RNA) characterized by molecular size of more than 100 bases are known in the art, and are disclosed, for example in U.S. Pat. Nos. 5,898,071, 5,705,628 and 6,534,262.

There is currently no quick and effective procedure for separating short nucleic acids (i.e., less than 100 nucleotides) of different lengths, and particularly no quick and effective method for differentiating between sRNA linked to one adaptor, unbound adaptors, sRNA linked to two adaptors and adaptor-adaptor dimers. Currently separation of the by-products from the ligation reactions is performed by electrophoresis on acrylamide gel, a cumbersome step which results in a loss of a significant proportion of the desired product and eliminates the possibility of using automated library preparation procedures.

### SUMMARY OF THE INVENTION

The present invention provides methods, kits and compositions for separation, identification, and isolation of short nucleic acids (i.e., less than 100 nucleotides) of different length. The invention further provides methods for preparation of small RNA libraries.

According to a first aspect, there is provided a method for separating a nucleic acid molecule of a desired length below 100 nucleotides from a solution comprising a nucleic acid molecules of the desired length and a nucleic acid molecule of a length at least 15 nucleotides shorter, the method comprising:
(a) obtaining a solution comprising nucleic acid molecules of multiple lengths;
(b) adding to the solution particles comprising a carboxyl-group coated surface, salt, polyalkylene glycol, and alcohol; and
(c) isolating the particles;
thereby separating a nucleic acid molecule of a desired length below 100 nucleotides from a solution comprising a nucleic acid molecules of the desired length and a nucleic acid molecule of a length at least 15 nucleotides shorter.

According to some embodiments, the polyalkylene glycol is polyethylene glycol (PEG). According to some embodiments, the alcohol is isopropanol. According to some embodiments, the salt is sodium chloride (NaCl).

According to some embodiments, the salt reaches a final concentration of between 0.8 and 1 molar. According to some embodiments, the final concentration of polyalkylene glycol is between 7.0% and 8.5%. According to some embodiments, the final concentration of polyalkylene glycol is between 7.5% and 8.0%. According to some embodiments, the final concentration of alcohol is between 67% minus 0.59% times the desired length in nucleotides and 75% minus 0.59% times the desired length in nucleotides. According to some embodiments, the final concentration of alcohol is between 70% minus 0.59% times the desired length in nucleotides and 74% minus 0.59% times the desired length in nucleotides. According to some embodiments, the final concentration of alcohol is about 73.7% minus 0.59% times the desired length in nucleotides.

According to some embodiments, the methods of the invention further comprise incubating the solution of step (b) for an amount of time sufficient for binding of the desired nucleic acid molecule to the particles prior to step (c).

According to some embodiments, the separating results in less than a 10% contamination by the nucleic acid molecule 15 nucleotides shorter than the desired length.

By another aspect, there is provided a method for separating a nucleic acid molecule of a desired length below 100 nucleotides from a solution comprising nucleic acid molecules of multiple lengths, the method comprising:
(a) combining in a reaction vessel particles comprising carboxyl group coated surfaces, sodium chloride, polyethylene glycol (PEG), isopropanol, and a solution comprising nucleic acid molecules comprising a first nucleic acid molecule having a desired length and a second nucleic acid molecule having a length of at least 15 bases shorter than the first nucleic acids molecule, to form a binding solution having concentrations of PEG and isopropanol suitable for selective binding of the first nucleic acid molecule to the particles; wherein
   (i) the length of the first nucleic acid molecule is at least 60 bases and the concentration of PEG and isopropanol is 7% -8.5% and 32% -41%, respectively;
   (ii) the length of the first nucleic acid molecule is at least 50 bases and the concentration of PEG and isopropanol is 7% -8.5% and 38% -45%, respectively;
   (iii) the length of the first nucleic acid molecule is at least 40 bases and the concentration of PEG and isopropanol is 7%-8.5% and 41%-50%, respectively; and
   (iv) the length of the first nucleic acid molecule is at least 30 bases and the concentration of PEG and isopropanol is 7% -8.5% and 45% -58%, respectively; and
   (v) the length of the first nucleic acid molecule is at least 20 bases and the concentration of PEG and isopropanol is 7% -8.5% and 49% -60%, respectively, and
(b) separating the particles;
thereby separating a nucleic acid molecule of a desired length below 100 nucleotides.

According to some embodiments, the particles are paramagnetic particles. According to some embodiments, the particles are separated or isolated from the solution by applying a magnetic field. According to some embodiments, the particles are separated or isolated by a method selected from the group of methods consisting of: applying vacuum filtration and centrifugation. According to some embodiments, the methods of the invention further comprise discarding supernatant from the reaction vessel. According to some embodiments, the methods of the invention further comprise washing the particles. According to some embodiments, the methods of the invention further comprise eluting the nucleic acid molecule of a desired length from the particles by applying an aqueous solution.

According to some embodiments, the nucleic acid molecule of a desired length is one of the following: a single-stranded nucleic acid molecule and a double-stranded nucleic acid molecule. According to some embodiments, the nucleic acid molecule of a desired length is a small RNA. According to some embodiments, the nucleic acid molecule of a desired length is a ligation product. According to some embodiments, the ligation product comprises a nucleic acid molecule ligated to at least one of the following: an oligonucleotide at the nucleic acid molecule's 3′ end, an oligonucleotide at the nucleic acid molecule's 5′ end, and an oligonucleotide at both ends.

According to some embodiments, at least one of the oligonucleotides comprises a nucleotide barcode. According to some embodiments, at least one of the oligonucleotides comprises a random sequence. According to some embodiments, the random sequence uniquely identifies the nucleic acid molecule. According to some embodiments, the random sequence distinguishes between an original nucleic acid molecule and amplified copies thereof. According to some embodiments, the solution comprising nucleic acid molecules is selected from: an outcome of a reverse transcription procedure, extracted cellular RNA, a cell lysate, an outcome of an amplification procedure, an outcome of a ligation procedure, and an outcome of a restriction enzyme digestion.

According to another aspect, there is provided a method for preparing a small RNA library, the method comprising:
(a) obtaining a first solution comprising RNA molecules shorter than 100 nucleotides and substantially depleted of RNA molecules longer than 100 nucleotides;
(b) removing from the first solution RNA longer than 40 nucleotides by adding to the first solution particles comprising a carboxyl-group coated surface, salt to a final concentration of between 0.8 and 1 molar, polyalkylene glycol to a final concentration of between 7 and 8.5%, and alcohol to a final concentration of between 41 and 49% and subsequently removing the particles;
(c) isolating from the first solution RNA longer than 19 nucleotides by adding to the first solution particles comprising a carboxyl-group coated surface, salt to a final concentration of between 0.8 and 1 molar, polyalkylene glycol to a final concentration of between 7 and 8.5%, and alcohol to a final concentration of between 53 and 60.0% and subsequently isolating the particles and optionally eluting the RNA longer than 19 nucleotides into a second solution,
(d) ligating a 3′ adapter to the isolated RNA longer than 19 nucleotides;
(e) isolating RNA ligated to a 3′ adapter by adding particles comprising a carboxyl-group coated surface, salt to a final concentration of between 0.8 and 1 molar, polyalkylene glycol to a final concentration of between 7 and 8.5%, and alcohol to a final concentration of between 45 and 52% and subsequently isolating the particles and optionally eluting the RNA ligated to a 3′ adapter into a third solution;
(f) ligating a 5′ adapter to the isolated RNA ligated to a 3′ adapter;
(g) isolating RNA ligated to a 3′ and 5′ adapter by adding particles comprising a carboxyl-group coated surface, salt to a final concentration of between 0.8 and 1 molar, polyalkylene glycol to a final concentration of between 7 and 8.5%, and alcohol to a final concentration of between 32 and 44% and subsequently isolating the particles;
thereby preparing a small RNA library.

According to some embodiments, the solution of step (a) is depleted of RNA molecules longer than 100 nucleotides by use of a kit for extraction of high molecular weight nucleic acids. According to some embodiments, the alcohol in step (b) is at a final concentration of about 44%. According to some embodiments, the alcohol in step (c) is at a final concentration of about 54.5%. According to some embodiments, the polyalkylene glycol is at a final concentration of about 7.78% and the alcohol is at a final concentration of about 54.5%.

According to some embodiments, the 3′ adapter is about 18 nucleotides long, and the alcohol in step (e) is at a final concentration of about 48%. According to some embodiments, the methods of the invention further comprise adding a blocking oligo to the isolated RNA after step (e).

According to some embodiments, the 5′ adapter is between 19 and 37 nucleotides long and the alcohol in step (g) is at a final concentration of between 35 and 38%. According to some embodiments, the 5′ adapter is about 27 nucleotides long and the alcohol in step (g) is at a final concentration of about 35%. According to some embodiments, the 5′ adapter comprises a barcode. According to some embodiments, the 5′ adapter comprises a random sequence. According to some embodiments, the random sequence uniquely identifies a RNA molecule and can distinguish between an RNA originally in the solution of step (a) and an amplified copy thereof. According to some embodiments, the 5′ adapter is 27 nucleotides long, and the alcohol is step (f) is at a final concentration of about 35%.

According to some embodiments, the methods of the invention further comprise eluting the isolated RNA longer than 56 nucleotides from the particles by applying an aqueous solution. According to some embodiments, the methods of the invention further comprise reverse transcribing the isolated RNA longer than 56 nucleotides into cDNA. According to some embodiments, the methods of the invention further comprise PCR amplifying the cDNA.

According to some embodiments, the polyalkylene glycol is PEG, the alcohol is isopropanol, and the salt is NaCl.

According to some embodiments, the methods of the invention further comprise washing the particles following every isolation.

By another aspect, there is provided a kit for isolating and separating nucleic acid molecules of a desired length below 100 nucleotides, the kit comprising:
(a) at least one of the following: (i) a table of efficiencies of binding of nucleic acid molecules of different lengths to particles comprising a carboxyl-group coated surface for a range of concentrations of PEG and isopropanol, and (ii) an equation for calculating ideal isopropanol and PEG concentration for binding a nucleic acid molecule to a carboxyl-group coated surface and
(b) at least one of the following: (i) particles comprising carboxyl group coated surfaces; (ii) PEG; and (iii) isopropanol.

According to some embodiments, the kits of the invention are for use in preparing a small RNA library, wherein the kit further comprises instruction for preparing a small RNA library and at least one of the following components: (i) 3′-oligonucleotides; (ii) 3′-oligonucleotides comprising an adenylated 5′ end; (iii) 5′-oligonucleotides; (iv) an oligonucleotide comprising a nucleotide barcode comprising a random sequence; (v) an RNA ligase; (vi) a reverse transcriptase; and (vii) a DNA polymerase.

### BRIEF DESCRIPTION OF THE DRAWINGS

FIG. 1: A scatter plot showing ideal concentrations of isopropanol for the separation of oligonucleotides of various lengths. A best fit line is given for the five data points.

FIGS. 2A-I: Tapestation traces of an input mix of two ssDNA oligonucleotides (2A, 2D, 2G), a right-side size-selection (2B, 2E, 2H) and a left-side size-selection (2C, 2F, 2I). Three different isopropanol conditions were employed for the right-side size-selection, 38% (2A-C), 41% (2D-F), and 44% (2G-I). The left-side size-selection was performed on the supernatant remaining after the right-side size-selection. Peak sizes and corresponding fragments areas are marked by lines; the left peak titled “lower” is a 25 nt size marker.

FIGS. 3A-B: (3A) A general scheme for preparation of a sRNA library for high-throughput sequencing from a low molecular weight (LMW) RNA fraction. (3B) A schematic view of integrating Unique Molecular Identifiers (UMIs) to sRNA library preparation.

FIGS. 4A-D: Libraries were prepared from 1 ug of a LMW RNA fraction extracted from C. elegans L4 stage (4A-B) and 1 ug of human brain total RNA (4C-D). (4A, 4C) Tapstation traces of the amplification products after 17 cycles of PCR amplification. (4B, 4D) Tapstation traces of the sRNA library after purification of the 17-cycle amplification product followed by SPRI double size selection using standard conditions, i.e. PEG only. sRNA library corresponding peaks are marked by an arrow. Peak sizes are marked by a line. The peaks titled “Lower” and “Upper” correspond to 25-nucleotide and 1500-nucleotide molecular size markers.

FIG. 5: A log-scale scatter plot comparing miRNA expression from two libraries constructed using either 1 ug or 100 ng from the same input material from L4 larval stage. Every dot in the plot represents a sequence count for a miRNA after sequences were collapsed based on 8N UMI. A regression line for all miRNAs is presented in the graph.

FIG. 6A-D: Dispersion plots generated by DESEQ package in R using estimate Dispersions function. Sequences aligned to each miRNA were counted and the variance of the three replicate samples was estimated. Each dot in the plot represents variance between the replicate samples for specific miRNA counts. Dispersion values are the variation between samples squared.

All plots are samples generated from L4 stage, including (6A) technical replicate samples dispersion estimated with collapsed reads, (6B) biological replicate samples dispersion estimated with collapsed reads, (6C) technical replicate samples dispersion estimated with non-collapsed reads, and (6D) biological replicate samples dispersion estimated with non-collapsed reads.

### DETAILED DESCRIPTION OF THE INVENTION

In the description that follows, a number of terms related to recombinant DNA technology are used. In order to provide a clear and consistent understanding of the specifications and claims, the following definitions are provided.

The term “nucleic acid” is well known in the art. A “nucleic acid” generally refers to a molecule (i.e., a single or double strand) of DNA, RNA or a derivative or analog thereof, comprising a nucleotide. The terms “nucleotide” and “base” as used interchangeably herein encompasses both nucleotides and ribonucleotides and include, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The terms “nucleic acid” and “nucleic acid molecule”, as used interchangeably herein, include, for example, single-stranded nucleic acid molecules such as single-stranded RNA (ssRNA) and single-stranded DNA (ssDNA), double-stranded nucleic acid molecules such as double-stranded RNA (dsRNA) and double-stranded DNA (dsDNA), small RNA, miRNA, siRNA, snoRNAs, snRNAs, tRNA, piRNA, tnRNA, small rRNA, hnRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides.

The term “small RNA” as used herein refers to short non-coding RNA molecules, including but not limited to microRNAs (miRNAs), small interfering RNAs (siRNAs), small nuclear RNAs (snRNAs), small nucleolar RNAs (snoRNAs), small temporal RNAs (stRNAs), antigene RNAs (agRNAs), piwi-interacting RNAs (piRNAs) and other short -regulatory nucleic acids.

As used herein, “particles” refer to solid phase carriers used to reversibly bind nucleic acid molecules. In some embodiments, particles include, but are not limited to, microparticles, fibers, beads and/or supports. In some embodiments, particles embody a variety of shapes that are either regular or irregular in form. In some embodiments, particles are used to reversibly bind nucleic acid molecules. The particles typically have sufficient surface area to permit efficient binding. The surface is typically coated with moieties possessing a functional group which reversibly binds nucleic acid molecules. One skilled in the art will appreciate that binding of nucleic acid molecules to the particles is dependent on the length of the nucleic acid molecules and is not dependent on the specific nucleic acid sequence.

In some embodiments, the functional group acts as a bio-affinity adsorbent for nucleic acid molecules precipitated by polyethylene glycol (PEG) or PEG and isopropanol. In some embodiments, the functional group is a carboxylic acid. In some embodiments, particles comprising a functional group-coated surface that reversibly binds nucleic acid molecules, include but are not limited to, amino-coated, carboxyl-coated and encapsulated carboxyl group-coated particles. Typically, the particles are of size that enables their separation from solution. Typically, the particles sizes range from about 0.1 micron mean diameter to about 100 micron mean diameter. Typically, the particles can be separated from a solution by methods known to those skilled in the art such as, but not limited to vacuum, filtration or centrifugation.

In some embodiments, the particles are paramagnetic particles. As used herein, the term “paramagnetic particles” refers to particles which respond to an external magnetic field but demagnetize when the field is removed. In some embodiments, the paramagnetic particles are efficiently separated from a solution using a magnet, and can be easily re-suspended without magnetically induced aggregation occurring. In some embodiments, paramagnetic particles can be separated from a solution using methods known to those skilled in the art such as, but not limited to vacuum, filtration or centrifugation. Suitable paramagnetic particles for use in the instant invention can be obtained for example from Bangs Laboratories Inc., Fishers, Inc., Beckman coulter, Inc and AMS Biotechnology.

As used herein the terms “separating”, “excluding”, “isolating” or “purifying” are used interchangeably, and are intended to mean that the material (e.g., nucleic acid molecules of a desired size) has been completely, substantially or partially separated, isolated, excluded or purified from other components present in the reaction vessel, e.g., membrane, proteins, nucleic acid molecules of un desired size.

As used herein, the term “oligonucleotide” refers to a short (e.g., no more than 100 bases), chemically synthesized single-stranded DNA or RNA molecule. In some embodiments, oligonucleotides are attached to the 5′ or 3′ end of a nucleic acid molecule, such as by means of ligation reaction. In some embodiments, oligonucleotide provides priming sequence that is used for reverse transcription, amplification and/or sequencing of the nucleic acid molecule. In some embodiments, oligonucleotides comprise sequences such as barcode or random sequences that are useful for identification of the origin of specific molecules or other applications.

As used herein, “enzymatic procedure” is any procedure performed by an enzyme on nucleic acid molecule(s) such as ligation procedure, reverse transcription procedure, amplification procedure, digestion procedure, dephosphorylation procedure, to name a few. An outcome of an enzymatic procedure comprises a desired product and by-products. As used herein, “byproducts of the enzymatic procedure” comprises nucleic acid molecules in which unintended enzymatic events have occurred and nucleic acid molecules in which not all of the intended enzymatic events have occurred during a reaction in which multiple nucleic acid molecules are present. The term “byproducts of the enzymatic procedure” as used herein, also includes nucleic acid molecules in which none of the intended enzymatic events have occurred.

As used herein, the term “byproduct of a ligation procedure” is a nucleic acid molecule which is formed by the unintended joining of two or more nucleic acid molecules or a nucleic molecule in which not all or none of the intended joining events have occurred during a reaction in which multiple nucleic acid molecules are present.

### Methods for Separating Nucleic Acid Molecules

The invention provides methods and kits for separating and/or isolating nucleic acid molecules having a length of no more than 100 bases from a mixture of nucleic acid molecules on the basis of size difference of at least 15 bases.

By one aspect, the invention provides a method for separating a nucleic acid molecule of a desired length below 100 nucleotides from a solution comprising a nucleic acid molecules of the desired length and a nucleic acid molecule of a length at least 15 nucleotides shorter, the method comprising:
(a) obtaining a solution comprising nucleic acid molecules of multiple lengths;
(b) adding to said solution particles comprising a carboxyl-group coated surface, salt, polyalkylene glycol, and alcohol; and
(c) isolating said particles;
   thereby separating a nucleic acid molecule of a desired length below 100 nucleotides from a solution comprising a nucleic acid molecules of the desired length and a nucleic acid molecule of a length at least 15 nucleotides shorter.

By another aspect, the invention provides a method for separating a nucleic acid molecule of a desired length below 100 nucleotides from a solution comprising a nucleic acid molecules of the desired length and a nucleic acid molecule of a length at least 15 nucleotides shorter, wherein said separating results in less than a 5% contamination by said nucleic acid molecule 15 nucleotides shorter than the desired length, the method comprising:
(a) obtaining a solution comprising nucleic acid molecules of multiple lengths;
(b) adding to said solution particles comprising a carboxyl-group coated surface, salt, polyalkylene glycol, and alcohol
(c) isolating said particles;
   thereby separating a nucleic acid molecule of a desired length below 100 nucleotides from a solution comprising a nucleic acid molecules of the desired length and a nucleic acid molecule of a length at least 15 nucleotides shorter with less than a 5% contamination.

By another aspect, the invention provides a method for separating a nucleic acid molecule of a desired length below 100 nucleotides from a solution comprising nucleic acid molecule, the method comprising:
(a) obtaining a solution comprising nucleic acid molecules;
(b) adding to said solution particles comprising a carboxyl-group coated surface, salt, polyalkylene glycol, and alcohol, wherein said salt reaches a final concentration of between 0.8 and 1 molar, said polyalkylene glycol reaches a final concentration of between 7 and 8.5%, and said alcohol reaches a concentration of between 67% minus 0.59% times said desired length in nucleotides and 75% minus 0.59% times said desired length in nucleotides;
(c) isolating said particles;
   thereby separating a nucleic acid molecule of a desired length below 100 nucleotides from a solution comprising nucleic acid molecules of multiple lengths.

By another aspect, the invention provides a method for separating a nucleic acid molecule of a desired length below 100 nucleotides from a solution comprising nucleic acid molecule, the method comprising:
a. obtaining a solution comprising nucleic acid molecules;
b. adding to said solution particles comprising a carboxyl-group coated surface, salt, polyalkylene glycol, and alcohol, wherein said salt reaches a final concentration of between 0.8 and 1 molar, said polyalkylene glycol reaches a final concentration of between 7 and 8.5%, and said alcohol reaches a concentration of between 1% to 60%.
c. isolating said particles;
   thereby separating a nucleic acid molecule of a desired length below 100 nucleotides from a solution comprising nucleic acid molecules of multiple lengths.

In some embodiments, the invention provides a method for isolating and/or excluding nucleic acid molecules of a desired length, the method comprising: combining in a reaction vessel particles, salt, polyalkylene glycol, alcohol, and a mixture of nucleic acid molecules comprising a first set of nucleic acid molecules having a length of no more than 100 bases long and a second set of nucleic acid molecules having a length of at least 15 bases shorter than the first set of nucleic acids molecules, to form a binding solution having concentration of polyalkylene glycol and alcohol suitable for selective binding of the first set of nucleic acid molecules to the particles; and separating the particles; thereby isolating and/or excluding the first set of nucleic acid molecules.

In some embodiments, the desired length is the length of the first set of nucleic acid molecules. In some embodiments, the nucleic acid of a desired length refers to the first set of nucleic acid molecules. In some embodiments, the desired length refers to the minimal number of bases of the nucleic acid molecules comprising the first set of nucleic acid molecules. In some embodiments, the desired length is any length above a specific threshold. In some embodiments, the desired length is any length above a specific threshold and not below a specific threshold. In some embodiments, the desired length is a plurality of lengths. As a non-limiting example, a desired length may be any nucleic acid longer than 60 nucleotides and thus may comprise molecules having various lengths including 60 bases, 61 bases, 62 bases, 63 bases, 64 bases, 65 bases, 70 bases and so on.

As used herein, the terms “multiple lengths” and “various lengths” refer to a mix of nucleic acid molecules with a plurality of lengths. In some embodiments, nucleic acids of multiple lengths all have a length not greater than 100 nucleotides.

As used herein, the term “set of nucleic acid molecules”, as in the “first set” and the “second set” of nucleic acid molecules, may encompass a plurality of nucleic acid molecules having an identical length as well as a plurality of nucleic acid molecules having a varying length. A designated length of the first set refers to the length of the shortest nucleic acid molecules of the first set. As a non-limiting example, a first set of 60 bases may comprise molecules having various lengths including 60 bases, 61 bases, 62 bases, 63 bases, 64 bases, 65 bases, 70 bases and so on. A designated length of the second set refers to the length of the longest nucleic acid molecules of the second set. As a non-limiting example, a second set of 45 bases may comprise molecules having various lengths including 45 bases, 44 bases, 40 bases, 32 bases, 25 bases and so on.

In another embodiment, the length of the second set of nucleic acid molecules includes molecules of various length having a maximal length of at least 15 bases shorter than the minimal length of molecules comprising the first set of nucleic acid molecules. The term “at least 15 bases shorter than the first set of nucleic acid molecules” relates to a difference of 15 bases from the shortest nucleic acid molecules of the first set. As a non-limiting example for embodiments wherein the first set of nucleic acid molecules is of at least 60 bases, the second set of molecules will comprise molecules having a length of 45 bases or shorter.

In some embodiments, more than 90% of the first set of nucleic acid molecules, or the nucleic acid molecules of a desired length, is bound to the particles. In some embodiments, more than 80% of the first set of nucleic acid molecules, or the nucleic acid molecules of a desired length, is bound to the particles. In some embodiments, more than 70% of the first set of nucleic acid molecules, or the nucleic acid molecules of a desired length, is bound to the particles. In some embodiments, more than 60% of the first set of nucleic acid molecules, or the nucleic acid molecules of a desired length, is bound to the particles. In some embodiments, more than 50% of the first set of nucleic acid molecules, or the nucleic acid molecules of a desired length, is bound to the particles. In some embodiments, more than 40% of the first set of nucleic acid molecules, or the nucleic acid molecules of a desired length, is bound to the particles. In some embodiments, more than 30% of the first set of nucleic acid molecules, or the nucleic acid molecules of a desired length, is bound to the particles. In some embodiments, more than 20% of the first set of nucleic acid molecules, or the nucleic acid molecules of a desired length, is bound to the particles. Each possibility represents a separate embodiment of the present invention.

As used herein, the percentage of input of a nucleic acid molecule of a desired length, or of the first set of molecules, that binds to the particles in a reaction is referred to as the “yield”. In some embodiments, the yield of the methods of the invention is at least 40%, 45%, 50%, 55%, 60%, 65%, 70%, 75%, 80%, 85%, 90%, 95%, 99% or 100%. Each possibility represents a separate embodiment of the present invention. As used herein, “input” refers to the amount of a nucleic acid molecule, or a set of molecules, that is present before a separation, isolation, or exclusion is performed.

Binding of the second set of nucleic acid molecules, or of nucleic acid molecules of a length at least 15 nucleotides shorter than the desired length is herein referred to as “contamination”. In some embodiments, less than 10%, 7.5%, 5%, 4%, 3%, 2%, or 1% of the second set of nucleic acid molecules is bound to the particles. Each possibility represents a separate embodiment of the present invention. In some embodiments, less than 10%, 7.5%, 5%, 4%, 3%, 2%, or 1% of undesired nucleic acid molecules is bound to the particles. Each possibility represents a separate embodiment of the present invention. In some embodiments, less than 10%, 7.5%, 5%, 4%, 3%, 2%, or 1% of nucleic acid molecules of a length at least 15 nucleotides shorter than the desired length is bound to the particles. Each possibility represents a separate embodiment of the present invention. In some embodiments, separating results in less than a 5% contamination by the nucleic acid molecule 15 nucleotides shorter than the desired length. In some embodiments, separating results in less than a 10% contamination by the nucleic acid molecule 15 nucleotides shorter than the desired length.

In some embodiments, the contamination resulting from the methods of the invention is not more than 10%, 7.5%, 5%, 4%, 3%, 2% or 1% of the total binding. Each possibility represents a separate embodiment of the present invention. In some embodiments, the contamination resulting from the methods of the invention is not more than 5% of the total binding.

In embodiments, wherein the difference in sizes of the two sets of nucleic acid molecules is smaller than 15 bases, or wherein the desired and undesired molecules have lengths within 15 bases, the concentration of polyalkylene glycol and alcohol suitable for selective binding of desired nucleic acid molecules may result in binding of a portion of undesired nucleic acid molecules as well (i.e., in contamination by undesired molecules). In some embodiments, higher selectivity may be achieved by compromising the binding efficiency of the first set, or desired molecules. In a non-limiting example, binding of 60% of nucleic acid molecules comprising the first set and 6% of nucleic acid molecules comprising the second set, is considered to be selective. In another non-limiting example, binding of 20% of nucleic acid molecules comprising the first set and 1% of nucleic acid molecules comprising the second set, is considered to be selective.

Typically, to allow binding of nucleic acid molecules to the particles, sufficient incubation time in the reaction vessel is needed e.g., incubation for at least 15 seconds, at least 30 seconds, at least 60 seconds, at least 2 minutes, at least 3 minutes, at least 4 minutes or at least 5 minutes. Each possibility represents a separate embodiment of the present invention. In some embodiments, the methods of the invention further comprise incubating a solution for an amount of time sufficient for binding of a desired nucleic acid molecule to the particles. In some embodiments of the methods of the invention every isolation or separation step comprises incubating the solution for an amount of time sufficient for binding of a nucleic acid molecule to the particles. In some embodiments of the methods of the invention at least one isolation or separation step comprises incubating the solution for an amount of time sufficient for binding of a nucleic acid molecule to the particles.

In some embodiments, following the incubation, the particles having nucleic acid molecules bound thereto can be separated from the binding solution by methods known to those skilled in the art such as, but not limited to vacuum, filtration, centrifugation and application of a magnetic field.

In some embodiments, the final concentration of polyalkylene glycol is between 6-9%, 6-8.5%, 6-8%, 6-7.5%, 6.5-9%, 6.5-8.5%, 6.5-8%, 6.5-7.5%, 7-9%, 7-8.5%, 7-8%, 7-7.5%, 7.5-9%, 7.5-8.5%, or 7.5-8%. Each possibility represents a separate embodiment of the present invention. In some embodiments, the final concentration of polyalkylene glycol is between 7.5 and 8.5%. In some embodiments, the final concentration of polyalkylene glycol is between 7.5 and 8.0%.

In some embodiments, the salt reaches a final concentration of between 0.5-2, 0.5-1.8, 0.5-1.6, 0.5-1.4, 0.5-1.2, 0.5-1.0, 0.5-0.9, 0.6-2, 0.6-1.8, 0.6-1.6, 0.6-1.4, 0.6-1.2, 0.6-1.0, 0.6-0.9, 0.7-2, 0.7-1.8, 0.7-1.6, 0.7-1.4, 0.7-1.2, 0.7-1.0, 0.7-0.9, 0.8-2, 0.8-1.8, 0.8-1.6, 0.8-1.4, 0.8-1.2, 0.8-1.0, or 0.8-0.9 molar. Each possibility represents a separate embodiment of the present invention. In some embodiments, the salt reaches a final concentration of between 0.8 and 1 molar.

In some embodiments, the polyalkylene glycol is polyethylene glycol (PEG). In some embodiments, the alcohol is isopropanol. In some embodiments, the salt is sodium chloride (NaCl). In some embodiments, the salt is NaCl and the NaCl reaches a final concentration of between 0.8 and 1 molar. In some embodiments, the polyalkylene glycol is PEG and the salt is NaCl and the final concentration of PEG is between 7.5 and 8.5%. In some embodiments, the polyalkylene glycol is PEG and the salt is NaCl and the final concentration of PEG is between 7.5 and 8.0%. In some embodiments, the polyalkylene glycol is PEG and the salt is NaCl, the final concentration of NaCl is between 0.9 and 0.93 molar and the final concentration of PEG is between 7.5 and 8.5%. In some embodiments, the polyalkylene glycol is PEG and the salt is NaCl, the final concentration of Nacl is between 0.9 and 0.93 molar and the final concentration of PEG is between 7.5 and 8.0%.

In some embodiments, the final concentration of alcohol is between 68% minus 0.59% times said desired length in nucleotides and 74% minus 0.59% times said desired length in nucleotides. In some embodiments, the final concentration of alcohol is between 69% minus 0.59% times said desired length in nucleotides and 74% minus 0.59% times said desired length in nucleotides. In some embodiments, the final concentration of alcohol is between 70% minus 0.59% times said desired length in nucleotides and 74% minus 0.59% times said desired length in nucleotides. In some embodiments, the final concentration of alcohol is between 71% minus 0.59% times said desired length in nucleotides and 74% minus 0.59% times said desired length in nucleotides. In some embodiments, the final concentration of alcohol is between 68% minus 0.59% times said desired length in nucleotides and 75% minus 0.59% times said desired length in nucleotides. In some embodiments, the final concentration of alcohol is between 69% minus 0.59% times said desired length in nucleotides and 75% minus 0.59% times said desired length in nucleotides. In some embodiments, the final concentration of alcohol is between 70% minus 0.59% times said desired length in nucleotides and 75% minus 0.59% times said desired length in nucleotides. In some embodiments, the final concentration of alcohol is between 71% minus 0.59% times said desired length in nucleotides and 75% minus 0.59% times said desired length in nucleotides. In some embodiments, the final concentration of alcohol is about 73.7% minus 0.59% times said desired length in nucleotides. In some embodiments, the final concentration of alcohol is not greater than 54.5. In some embodiments, the final concentration of alcohol is only greater than 54.5 when the final concentration of polyalkylene glycol is less than 7.5.

In some embodiments, the polyalkylene glycol is PEG, the salt is NaCl, the alcohol is isopropanol and the final concentration of isopropanol is between 67% minus 0.59% times said desired length in nucleotides and 75% minus 0.59% times said desired length in nucleotides. In some embodiments, the polyalkylene glycol is PEG, the salt is NaCl, the alcohol is isopropanol and the final concentration of isopropanol is between70% minus 0.59% times said desired length in nucleotides and 74% minus 0.59% times said desired length in nucleotides. In some embodiments, the polyalkylene glycol is PEG, the salt is NaCl, the alcohol is isopropanol and the final concentration of isopropanol is about 73.7% minus 0.59% times said desired length in nucleotides.

In some embodiments, the invention provides a method for isolating and/or excluding nucleic acid molecules of desired length, the method comprising: combining in a reaction vessel particles, sodium chloride (NaCl), polyethylene glycol (PEG), isopropanol, and a mixture of nucleic acid molecules comprising a first set of nucleic acid molecules having a length of no more than 100 bases long and a second set of nucleic acid molecules having a length of at least 15 bases shorter than the first set of nucleic acid molecules, to form a binding solution having concentration of PEG and isopropanol suitable for selective binding of the first set of nucleic acid molecules to the particles; wherein
(i) the length of the first set of nucleic acid molecules is at least 60 bases and the concentration of PEG and isopropanol is 7%-8.5% and 32%-41%, respectively;
(ii) the length of the first set of nucleic acid molecules is at least 50 bases and said concentration of PEG and isopropanol is 7%-8.5% and 38%-45%, respectively;
(iii) the length of the first set of nucleic acid molecules is at least 40 bases and the concentration of PEG and isopropanol is 7%-8.5% and 41-50%, respectively;
(iv) the length of the first set of nucleic acid molecules is at least 30 bases and the concentration of PEG and isopropanol is 7%-8.5% and 45%-54%, respectively;
(v) the length of the first set of nucleic acid molecules is at least 20 bases and the concentration of PEG and isopropanol is 7%-8.5% and 51%-55%, respectively; and separating the particles; thereby isolating and/or excluding the first set of nucleic acid molecules.

As exemplified herein below in Table 1, a higher concentration of isopropanol without changing PEG concentrations results in binding of shorter nucleic acid molecules to the particles.

In some embodiments of the invention, the length of the first set of nucleic acid molecules is at least 60 bases, the concentration of PEG is 6.5%-8.5%, or 7%-8%, or about 7.5% and the concentration of isopropanol is 25%-41%, or 26%-40%, or 27%-39%, or 28%-38%, or 29%-37%, or 30%-36%, or 31%-35%, or 32%-34%. Each possibility represents a separate embodiment of the invention.

In some embodiments of the invention, the length of the first set of nucleic acid molecules is at least 50 bases, the concentration of PEG is 6.5%-8.5%, or 7%-8%, or about 7.5% and the concentration of isopropanol is 37%-47%, or 38%-46%, or 39%-45%, or 40%-44%, or 41%-42%. Each possibility represents a separate embodiment of the invention.

In some embodiments of the invention, the length of the first set of nucleic acid molecules is at least 40 bases, the concentration of PEG is 6.5%-8.5%, or 7%-8%, or about 7.5% and the concentration of isopropanol is 40%-51%, or 41%-50%, or 42%-49%, or 43%-48%, or 44%-47%. Each possibility represents a separate embodiment of the invention.

In some embodiments of the invention, the length of the first set of nucleic acid molecules is at least 30 bases the concentration of PEG is 6.5%-8.5%, or 7%-8%, or about 7.5% and the concentration of isopropanol is 43%-55%, or 44%-54%, or 45%-55%, or 46%-54%, or 47%-53%, or 48%-53%, or 49%-52%, or 50%-51%. Each possibility represents a separate embodiment of the invention. In some embodiments, the concentration of isopropanol is at least 43%, or at least 43%, or at least 44%, or at least 45%, or at least 46%, or at least 47%, or at most 50%, or at most 52%, or at most 55%. Each possibility represents a separate embodiment of the invention.

In some embodiments of the invention, the length of the first set of nucleic acid molecules is at least 20 bases the concentration of PEG is 6.5%-8.5%, or 7%-8%, or about 7.5% and the concentration of isopropanol is 48%-57%, or 49%-56%, or 50%-55%, or 51%-54%, or 52%-53%. Each possibility represents a separate embodiment of the invention.

In some embodiments, the concentration of isopropanol is at least 50%, or at least 51%, or at least 52%, or at least 53%, or at least 55%, or at most 50%, or at most 55%, or at most 56%, or at most 57%, or at most 58%. Each possibility represents a separate embodiment of the invention.

In some embodiments, the particles are paramagnetic particles. In some embodiments, the particles are separated or isolated from the solution by applying a magnetic field. In some embodiments, the particles are separated or isolated by a method selected from the group of methods consisting of: applying vacuum filtration and centrifugation.

In some embodiments, following the step of separating the particles from the binding solution, the remaining supernatant (i.e., binding solution from which the particles and the nucleic acid molecules bound thereto were removed) may be discharged, thereby leaving the particles bound to nucleic acid molecules in the vessel. In some embodiments, the supernatant comprising the second set of nucleic acids can be transferred to a new tube and particles, polyalkylene glycol and alcohol are added to form a binding solution suitable for binding to particles of all or some of the lengths comprising the former second set of nucleic acids.

In some embodiments, the method further comprises a step of washing the particles at least once with a washing buffer, in order to remove unbound nucleic acid molecules and other components (e.g., cell components, salt) from the reaction vessel. In some embodiments, the methods of the invention further comprise washing the particles. In some embodiments, the washing buffer is any suitable washing buffer capable of removing unbound nucleic acid molecules and other components without causing detaching of bound nucleic acid molecules from the particles, known to a person with skilled in the art. In some embodiments, the washing buffer comprises alcohol. In some embodiments, the alcohol is ethanol. The concentration of ethanol in the washing buffer depends on the length of the first set of nucleic acid molecules (e.g., higher concentration of ethanol is required for shorter length of the first set of nucleic acid molecules). In some embodiments, the concentration of ethanol is more than 70%. In some embodiments, the concentration of ethanol equals 85%. In some embodiments, the concentration of ethanol is more than 85%.

In some embodiments, the methods of the invention further comprise eluting nucleic acid molecules from the particles, i.e., detaching nucleic acid molecules from the particles by contacting the particles with a suitable elution buffer. In some embodiments, the elution buffer is an aqueous buffer. The elution buffer may be any aqueous solution in which the molarity of salt, polyalkylene glycol and alcohol are below the concentrations required for binding of nucleic acid molecules to the particles. Examples of elution buffers include, but are not limited to, water, TRIS-HCl (10 millimolar (mM)). In other embodiments, the subsequent enzymatic reactions are performed without eluting the nucleic acid molecules.

In some embodiments, the eluted nucleic acid molecules are subsequently separated from the particles by using methods known to those skilled in the art such as, but not limited to vacuum, filtration centrifugation or magnetic separation. In some embodiments, an eluate comprising the isolated first set of nucleic acid molecules or the isolated desired molecules is produced. In some embodiments, the subsequent enzymatic reactions are performed without eluting the nucleic acid molecules from the particles and separating the eluate from the particles. In some embodiments, the reaction components are added directly into a vessel after washing the particles.

In some embodiments, the mixture of nucleic acid molecules is selected from, but not limited to: an outcome of a reverse transcription procedure comprising a mixture of single stranded DNA molecules, an outcome of amplification procedure, comprising a mixture of DNA molecules, an outcome of a ligation procedure comprising a mixture of ligated and un-ligated nucleic acid molecules, an outcome of in-vitro transcription procedure comprising a mixture of RNA molecules, an outcome of a restriction enzyme digestion comprising a mixture of DNA molecules and a cell lysate, which is a result of disrupting cells containing DNA and/or RNA and an extracted cellular RNA.

In some embodiments, the first set of nucleic acid molecules, or the nucleic acid molecule of a desired length, comprises ribonucleotides. In some embodiments, the first set of nucleic acid molecules, or the nucleic acid molecule of a desired length, comprises small RNAs. In some embodiments, the first set of nucleic acid molecules, or the nucleic acid molecule of a desired length, comprises microRNAs.

In some embodiments, the desired nucleic acid molecule is one of the following: a single-stranded nucleic acid molecule and a double-stranded nucleic acid molecule. In some embodiments, the nucleic acid molecule of a desired length is small RNA. In some embodiments, the nucleic acid molecule of a desired length is a ligation product. In some embodiments, the ligation product comprises a nucleic acid molecule ligated to at least one of the following: an oligonucleotide at said nucleic acid molecule's 3′ end, an oligonucleotide at said nucleic acid molecule's 5′ end, and an oligonucleotide at both ends. In some embodiments, the oligonucleotides at both ends are different oligonucleotides. In some embodiments, the oligonucleotides are adapters. In some embodiments, the ligation product with an oligonucleotide at both ends is an RNA ligated to a 3′ and a 5′ adapter.

In some embodiments, the oligonucleotide comprises a random sequence. In some embodiments, the oligonucleotide comprises a nucleotide barcode. In some embodiments, the barcode comprises a random sequence. In embodiments wherein an oligonucleotide comprising a random sequence is ligated to nucleic acid molecules, individual nucleic acid molecules are subsequently marked by a different/specific random sequence. The random sequence may then be used to distinguish between original nucleic acid molecules (each having a different random sequence) and amplified copies thereof (i.e., the copies of an original nucleic acid molecule having the same random sequence). In some embodiments, the random sequence uniquely identifies the nucleic acid molecule. In some embodiments, the random sequence distinguishes between an original nucleic acid molecule and amplified copies thereof.

Oligonucleotides comprising random sequence may be particularly useful for quantifying small RNA cellular content. Each one of the small RNA molecules is present in the cell in many copies at the same time, all the copies are amplified by PCR prior to sequencing. PCR amplification is non-linear and its effectiveness depends on the given sequence, causing quantification bias. Labeling of each individual small RNA molecule with a different random sequence prior to amplification, enables distinguishing between original copies of molecule and their amplification products, augmenting the reliability of the findings.

In some embodiments, the solution comprising nucleic acid molecules is selected from: an outcome of a reverse transcription procedure, extracted cellular RNA, a cell lysate, an outcome of an amplification procedure, an outcome of a ligation procedure, and an outcome of a restriction enzyme digestion.

In some embodiments, a solution comprising nucleic acid molecules can be any aqueous solution, such as, but not limited to a mixture containing DNA, RNA and derivatives thereof. In some embodiments, the solution comprising nucleic acid molecules also contains other components, such as other biomolecules, inorganic compounds and organic compounds (e.g., agarose, enzymes, DTT, Na Azide).

In some embodiments, the methods for isolating and/ or excluding nucleic acid molecules of desired length are utilized for preparation of small RNA libraries.

### Methods for Preparing a Small RNA Library

By another aspect, there is provided a method for preparing a small RNA library, the method comprising:
(a) obtaining a solution comprising RNA molecules shorter than 100 nucleotides and substantially depleted of RNA molecules longer than 100 nucleotides;
(b) removing from the solution RNA longer than 40 nucleotides by adding to the solution particles comprising a carboxyl-group coated surface, salt to a final concentration of between 0.8 and 1 molar, polyalkylene glycol to a final concentration of between 7 and 8.5%, and alcohol to a final concentration of between 41 and 49% and subsequently removing the particles;
(c) isolating from the solution RNA longer than 19 nucleotides by adding to the solution particles comprising a carboxyl-group coated surface, salt to a final concentration of between 0.8 and 1 molar, polyalkylene glycol to a final concentration of between 7 and 8.5%, and alcohol to a final concentration of between 53.0 and 60% and subsequently isolating the particles and optionally eluting the RNA longer than 19 nucleotides into a second solution,
(d) ligating a 3′ adapter to the isolated RNA longer than 19 nucleotides;
(e) isolating from the second solution RNA ligated to a 3′adapter by adding particles comprising a carboxyl-group coated surface, salt to a final concentration of between 0.8 and 1 molar, polyalkylene glycol to a final concentration of between 7 and 8.5%, and alcohol to a final concentration of between 45 and 52% and subsequently isolating the particles and optionally eluting the RNA ligated to a 3′ adapter into a third solution;
(f) ligating a 5′ adapter to the isolated RNA ligated to a 3′ adapter;
(g) isolating from the third solution RNA ligated to a 3′ and 5′ adapter by adding particles comprising a carboxyl-group coated surface, salt to a final concentration of between 0.8 and 1 molar, polyalkylene glycol to a final concentration of between 7 and 8.5%, and alcohol to a final concentration of between 32 and 44% and subsequently isolating the particles.
thereby preparing a small RNA library.

By another aspect, there is provided a method for preparing a small RNA library, the method comprising:
(a) obtaining a first solution comprising RNA molecules shorter than 100 nucleotides and substantially depleted of RNA molecules longer than 100 nucleotides;
(b) removing from said first solution RNA longer than 40 nucleotides by adding to said first solution particles comprising a carboxyl-group coated surface, salt to a final concentration of between 0.8 and 1 molar, polyalkylene glycol to a final concentration of between 7 and 8.5%, and alcohol to a final concentration of between 41 and 49% and subsequently removing said particles;
(c) isolating from said first solution RNA longer than 19 nucleotides by adding to said first solution particles comprising a carboxyl-group coated surface, salt to a final concentration of between 0.8 and 1 molar, polyalkylene glycol to a final concentration of between 7 and 8.5%, and alcohol to a final concentration of between 53 and 60.0% and subsequently isolating said particles and optionally eluting said RNA longer than 19 nucleotides into a second solution,
(d) ligating a 3′ adapter to said isolated RNA longer than 19 nucleotides;
(e) isolating RNA ligated to a 3′ adapter by adding particles comprising a carboxyl-group coated surface, salt to a final concentration of between 0.8 and 1 molar, polyalkylene glycol to a final concentration of between 7 and 8.5%, and alcohol to a final concentration of between 45 and 52% and subsequently isolating said particles and optionally eluting said RNA ligated to a 3′ adapter into a third solution;
(f) ligating a 5′ adapter to said isolated RNA ligated to a 3′ adapter;
(g) isolating said RNA ligated to a 3′ and 5′ adapter by adding particles comprising a carboxyl-group coated surface, salt to a final concentration of between 0.8 and 1 molar, polyalkylene glycol to a final concentration of between 7 and 8.5%, and alcohol to a final concentration of between 32 and 44% and subsequently isolating said particles;
thereby preparing a small RNA library.

In some embodiments of the method, the polyalkylene glycol is PEG, the alcohol is isopropanol and the salt is NaCl.

In some embodiments, the substantially depleted solution has had removed at least 50%, 60%, 70%, 80%, 90%, 95%, 99%, or 100% of the RNA molecules longer than 100 nucleotides. In some embodiments, the solution of step (a) is depleted of RNA molecules longer than 100 nucleotides by use of a kit for extraction of high molecular weight nucleic acids. Such kits are well known in the art, and include columns for isolation of RNA molecules which do not have the ability to bind molecules shorter than 100 nucleotides. In some embodiments, the depletion is carried out by addition of particles, salt and polyalkylene glycol without the addition of an alcohol or isopropanol. In some embodiments, the method for preparation of small RNA library comprises a preliminary step of separating low molecular weight RNA molecules (i.e. molecules shorter than 100 nucleotides) from longer nucleic acid molecules and other biomolecules and cell components such as proteins and lipids.

In some embodiments, the final concentration of PEG of any of the steps of the methods of the invention is between 6-9%, 6-8.5%, 6-8%, 6-7.5%, 6.5-9%, 6.5-8.5%, 6.5-8%, 6.5-7.5%, 7-9%, 7-8.5%, 7-8%, 7-7.5%, 7.5-9%, 7.5-8.5%, or 7.5-8%. Each possibility represents a separate embodiment of the present invention. In some embodiments, the final concentration of polyalkylene glycol for all the steps of the method is between 7.5 and 8.0%. In some embodiments of any step of the invention the PEG is at a final concentration of about 7.5.

In some embodiments, the alcohol in step (b) is at a final concentration between 41 and 49%, 41 and 48%, 41 and 47%, 41 and 46%, 41 and 45%, 42 and 49%, 42 and 48%, 42 and 47%, 42 and 46%, 42 and 45%, 43 and 49%, 43 and 48%, 43 and 47%, 43 and 46%, or 43 and 45%. Each possibility represents a separate embodiment of the present invention. In some embodiments, the alcohol in step (b) is at a final concentration between 41 and 49%. In some embodiments, the alcohol in step (b) is isopropanol and is at a final concentration between 41 and 49%. In some embodiments, the alcohol in step (b) is at a final concentration of about 44%. In some embodiments, the alcohol in step (b) is isopropanol and is at a final concentration of about 44%.

In some embodiments, the alcohol in step (c) is at a final concentration of between 53 and 54.5%, 53.1 and 54.5%, 53.2 and 54.5%, 53.3 and 54.5%, 53.4 and 54.5%, 53.5 and 54.5%, 53.6 and 54.5%, 53.7 and 54.5%, 53.8 and 54.5%, 53.9 and 54.5%, 54 and 54.5%., 53 and 55%, 53.1 and 55%, 53.2 and 55%, 53.3 and 55%, 53.4 and 55%, 53.5 and 55%, 53.6 and 55%, 53.7 and 55%, 53.8 and 55%, 53.9 and 55%, 54 and 55%, 53 and 56%, 53.1 and 56%, 53.2 and 56%, 53.3 and 56%, 53.4 and 56%, 53.5 and 56%, 53.6 and 56%, 53.7 and 56%, 53.8 and 56%, 53.9 and 56%, 54 and 56%, 53 and 57%, 53.1 and 57%, 53.2 and 57%, 53.3 and 57%, 53.4 and 57%, 53.5 and 57%, 53.6 and 57%, 53.7 and 57%, 53.8 and 57%, 53.9 and 57%, 54 and 57%, 53 and 58%, 53.1 and 58%, 53.2 and 58%, 53.3 and 58%, 53.4 and 58%, 53.5 and 58%, 53.6 and 58%, 53.7 and 58%, 53.8 and 58%, 53.9 and 58%, 54 and 58%, 53 and 59%, 53.1 and 59%, 53.2 and 59%, 53.3 and 59%, 53.4 and 59%, 53.5 and 59%, 53.6 and 59%, 53.7 and 59%, 53.8 and 59%, 53.9 and 59%, 54 and 59%, 53 and 60%, 53.1 and 60%, 53.2 and 60%, 53.3 and 60%, 53.4 and 60%, 53.5 and 60%, 53.6 and 60%, 53.7 and 60%, 53.8 and 60%, 53.9 and 60%, or 54 and 60%. Each possibility represents a separate embodiment of the present invention. In some embodiments, the alcohol in step (c) is at a final concentration of between 53.8 and 54.5. In some embodiments, the alcohol in step (c) is isopropanol and is at a final concentration of between 53.8 and 54.5. In some embodiments, the alcohol is at a final concentration of about 54.5%. In some embodiments, the alcohol is isopropanol and is at a final concentration of about 54.5%. In some embodiments, the polyalkylene glycol is at a final concentration of about 7.78%. In some embodiments, the polyalkylene glycol is PEG and is at a final concentration of about 7.78%. In some embodiments, the alcohol is isopropanol and is at a final concentration of about 54.5% and the polyalkylene glycol is PEG and is at a final concentration of about 7.78%.

In some embodiments, the 3′ adapter is about 18 nucleotides long. In some embodiments, the 3′ adapter is between 18 and 27 nucleotides long. In some embodiments, the 3′ adapter is about 18 nucleotides long, and the alcohol in step (e) is at a final concentration of about 48%. In some embodiments, the 3′ adapter is between 18 and 27 nucleotides long and the alcohol in step (e) is at a final concentration of between 43-48%. In some embodiments, the 3′ adapter is between 18 and 27 nucleotides long and the alcohol in step (e) is at a final concentration of about 48%.

In some embodiments, the alcohol in step (e) is at a final concentration between 45 and 54.5%, 45 and 54%, 45 and 53%, 45 and 52%, 45 and 51%, 45 and 50%, 46 and 54.5%, 46 and 54%, 46 and 53%, 46 and 52%, 46 and 51%, 46 and 50%, 47 and 54.5%, 47 and 54%, 47 and 53%, 47 and 52%, 47 and 51%, or 47 and 50%. Each possibility represents a separate embodiment of the present invention. In some embodiments, the alcohol in step (e) is at a final concentration between 45 and 54.5%. In some embodiments, the alcohol in step (e) is isopropanol and is at a final concentration between 45 and 54.5%. In some embodiments, the alcohol in step (e) is at a final concentration of about 48%. In some embodiments, the alcohol in step (e) is isopropanol and is at a final concentration of about 48%.

In some embodiments, the methods of the invention further comprise adding a blocking oligo to the isolated RNA after step (d). A blocking oligo refers to an oligonucleotide than binds to the 3′ adapter and blocks it from being a template for further reaction. One skilled in the art will understand that small contamination of the un-ligated adapter is possible, and addition of a blocking oligo decreases the creation of this undesired by-product.

In some embodiments, the 5′ adapter is between 19 and 35 nucleotides long. In some embodiments, the 5′ adapter comprises a barcode. In some embodiments, the 5′ adapter comprises a random sequence. In some embodiments, the barcode comprises a random sequence. In some embodiments, the random sequence uniquely identifies a RNA molecule and can distinguish between an RNA originally in the solution of step (a) and an amplified copy thereof. In some embodiments, the 5′ adapter is 27 nucleotides long, and the alcohol is step (g) is at a final concentration between 35.0 and 38.0%. In some embodiments, the 5′ adapter is between 19 and 35 nucleotides long and the alcohol is step (f) is between 29 and 38%.

In some embodiments, the alcohol is step (g) is at a final concentration between 29 and 54.5%, 30 and 54.5%, 31 and 54.5%, 32 and 54.5%, 33 and 54.5%, 34 and 54.5%, 35 and 54.5%, 29 and 44%, 30 and 44%, 31 and 44%, 32 and 44%, 33 and 44%, 34 and 44%, 35 and 44%, 29 and 43%, 30 and 43%, 31 and 43%, 32 and 43%, 33 and 43%, 34 and 43%, 35 and 43%, 29 and 42%, 30 and 42%, 31 and 42%, 32 and 42%, 33 and 42%, 34 and 42%, 35 and 42%, 29 and 41%, 30 and 41%, 31 and 41%, 32 and 41%, 33 and 41%, 34 and 41%, 35 and 41%, 29 and 40%, 30 and 40%, 31 and 40%, 32 and 40%, 33 and 40%, 34 and 40%, 35 and 40%, 29 and 39%, 30 and 39%, 31 and 39%, 32 and 39%, 33 and 39%, 34 and 39%, 35 and 39%, 29 and 38%, 30 and 38%, 31 and 38%, 32 and 38%, 33 and 38%, 34 and 38%, 35 and 38%, 29 and 37%, 30 and 37%, 31 and 37%, 32 and 37%, 33 and 37%, 34 and 37%, 35 and 37%, 29 and 36%, 30 and 36%, 31 and 36%, 32 and 36%, 33 and 36%, 34 and 36%, or 35 and 36%. Each possibility represents a separate embodiment of the present invention. In some embodiments, the alcohol is step (g) is at a final concentration between 32 and 44%.

In some embodiments, the 5′ adapter is 27 nucleotides long, and the alcohol is step (g) is at a final concentration between 29 and 38%, 30 and 38%, 31 and 38%, 32 and 38%, 33 and 38%, 34 and 38%, 35 and 38%, 29 and 37%, 30 and 37%, 31 and 37%, 32 and 37%, 33 and 37%, 34 and 37%, 35 and 37%, 29 and 36%, 30 and 36%, 31 and 36%, 32 and 36%, 33 and 36%, 34 and 36%, or 35 and 36%. Each possibility represents a separate embodiment of the present invention. In some embodiments, the 5′ adapter is 27 nucleotides long, and the alcohol is step (g) is at a final concentration of about 35%.

In some embodiments, the methods of the invention further comprise washing the particles following every isolation. In some embodiments, the methods of the invention further comprise eluting the isolated bound RNA ligated to a 3′ and 5′ adapter from the particles by applying an aqueous solution. In some embodiments, all elution steps are performed with an aqueous solution. It will be understood by one skilled in the art, that when the beads are to be discarded there is no need to wash them.

In some embodiments, the methods of the invention further comprise reverse transcribing the isolated RNA ligated to a 3′ and a 5′ adapter into cDNA. In some embodiments, the methods of the invention further comprise PCR amplifying the cDNA. It will be understood that reactions such as reverse transcription and PCR amplification can be performed directly on the beads or in a solution following elution.

One skilled in the art will appreciate that enzymatic reactions such as ligation, reverse transcription and amplification may be performed directly on the nucleic acid molecules bound to particles. Therefore, the entire procedure starting from step (c) can be performed in a single tube.

### Kits

By another aspect, there is provided a kit for isolating and separating nucleic acid molecules of a desired length below 100 nucleotides, the kit comprising:
(a) at least one of the following: (i) a table of efficiencies of binding of nucleic acid molecules of different lengths to particles comprising a carboxyl-group coated surface for a range of concentrations of PEG and isopropanol, and (ii) an equation for calculating ideal isopropanol and PEG concentration for binding a nucleic acid molecule to a carboxyl-group coated surface and
(b) at least one of the following: (i) particles comprising carboxyl group coated surfaces; (ii) PEG; and (iii) isopropanol.

In some embodiments, the kit of the invention is for use in preparing a small RNA library, wherein the kit further comprises instruction for preparing a small RNA library and at least one of the following components: (i) 3′-oligonucleotides; (ii) 3′- oligonucleotides comprising an adenylated 5′ end; (iii) a blocking oligo; (iv) 5′-oligonucleotides; (v) an oligonucleotide comprising a nucleotide barcode comprising a random sequence; (vi) an RNA ligase; (vii) a reverse transcriptase; and (viii) a DNA polymerase.

In some embodiments, the instruction for preparing a small RNA library comprise:
(a) obtaining a solution comprising RNA molecules shorter than 100 nucleotides and substantially depleted of RNA molecules longer than 100 nucleotides;
(b) removing from the solution RNA longer than 40 nucleotides by adding to the solution particles comprising a carboxyl-group coated surface, salt to a final concentration of between 0.8 and 1 molar, polyalkylene glycol to a final concentration of between 7 and 8.5%, and alcohol to a final concentration of between 41 and 49% and subsequently removing the particles;
(c) isolating from the solution RNA longer than 19 nucleotides by adding to the solution particles comprising a carboxyl-group coated surface, salt to a final concentration of between 0.8 and 1 molar, polyalkylene glycol to a final concentration of between 7 and 8.5%, and alcohol to a final concentration of between 53.8 and 60% and subsequently isolating the particles and eluting the RNA longer than 19 nucleotides into a second solution,
(d) ligating a 3′ adapter to the isolated RNA longer than 19 nucleotides;
(e) isolating from the second solution RNA ligated to a 3′adapter by adding particles comprising a carboxyl-group coated surface, salt to a final concentration of between 0.8 and 1 molar, polyalkylene glycol to a final concentration of between 7 and 8.5%, and alcohol to a final concentration of between 45.0 and 52% and subsequently isolating the particles and optionally eluting the RNA ligated to a 3′ adapter into a third solution;
(f) ligating a 5′ adapter to the isolated RNA ligated to a 3′ adapter;
(g) isolating from the third solution RNA ligated to a 3′ and 5′ adapter by adding particles comprising a carboxyl-group coated surface, salt to a final concentration of between 0.8 and 1 molar, polyalkylene glycol to a final concentration of between 7 and 8.5%, and alcohol to a final concentration of between 34 and 42% and subsequently isolating the particles.

In some embodiments, the instruction for preparing a small RNA library comprise any of the methods of the invention.

In some embodiments, the invention provides at least one of the following components: (i) a table of efficiencies of binding of nucleic acid molecules of different lengths to particles comprising a carboxyl-group coated surface for a range of concentrations of PEG and alcohol, and (b) an equation for calculating ideal alcohol and PEG concentration for binding a nucleic acid molecule to a carboxyl-group coated surface and at least one of the following components: (i) particles having carboxyl group coated surface (ii) PEG (iii) isopropanol (iv) NaCl (v) 3′-oligonucleotide (vi) 3′-oligonucleotide comprising adenylated 5′ end (vii) 5′-oligonucleotide (viii) an oligonucleotide comprising a nucleotide barcode (ix) a blocking peptide (x) a RNA ligase (xi) a reverse transcriptase (xii) a DNA polymerase (xiii) a protocol for preparation of small RNA libraries.

In some embodiments, the kit is a kit for isolating and/or excluding of nucleic acid molecules having a length of no more than 100 bases long.

In some embodiments, the invention provides a kit comprising at least one of the following components (i) a table of binding efficiencies (e.g., percentage of binding) of nucleic acid molecules of varying lengths to particles, for varying concentrations of polyalkylene glycol and alcohol (e.g., table 1) and (ii) an equation for calculating ideal isopropanol and PEG concentration for binding a nucleic acid molecule to a carboxyl-group coated surface. A skilled artisan will appreciate that the kit may comprise a table of many kinds, such as a table listing efficiencies of binding of nucleic acid molecules of varying lengths to particles for specific concentrations of polyalkylene glycol and alcohol, or a table listing efficiencies of binding of nucleic acid molecules of a specific length to particles at varying concentrations of polyalkylene glycol, alcohol or both. Such tables and equations can be utilized to select suitable concentrations of polyalkylene glycol and alcohol for selective binding of a first set of nucleic acid molecules to particles.

In other embodiments, said kit further comprises particles comprising carboxyl group coated surfaces. In some embodiments, the kit is useful for separating nucleic acid molecules of less than 100 bases from nucleic acid molecules that are at-least 15 bases shorter. In some embodiments, the kit is useful for preparation of small RNA libraries. In some embodiments, the kit further comprises a solution comprising Isopropanol, sodium chloride (NaCl) and/or PEG. In some embodiments, the kit further comprises one or more components selected from (i) 3′-oligonucleotide (ii) 3′-oligonucleotide comprising adenylated 5′ (iii) a blocking oligo (iv) 5′-oligonucleotide (v) an oligonucleotide comprising a random sequence (vi) RNA ligase (vii) a reverse transcriptase (viii) DNA polymerase (ix) a protocol for preparation of small RNA libraries.

A skilled artisan will appreciate that the methods and kits of the invention can be automated, such as to isolate and/or exclude nucleic acid molecules having a length of less than 100 basses or to prepare small RNA library for HTS.

In the application, unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization - A Laboratory Course Manual” CSHL Press (1996); all of which are incorporated by reference. Other general references are provided throughout this document.

As used herein, the term “about” when combined with a value refers to plus and minus 10% of the reference value. For example, a length of about 1000 nanometers (nm) refers to a length of 1000 nm+-100 nm

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

The following examples serve to illustrate certain embodiments and aspects of the present invention and are not to be construed as limiting the scope thereof. Those skilled in the art will appreciate that many changes could be made in the specific embodiments disclosed herein while still obtaining an identical or similar result.

### Materials and Methods

C. elegans Growth and Synchronization

Wildtype C. elegans strain Bristol N2, was used in this study and was maintained on OP50-seeded enriched plates at 20° C. Embryos were isolated from gravid N2 adults by treatment with sodium hypochlorite solution to dissolve animals of all stages but embryos. To obtain synchronized L4 worms, embryos were incubated in M9 media without food at 20° C. for 24h. Hutched synchronized L1 were grown on OP50-seeded Enriched plates at 20° C. until they reached L4 larval stage.

### RNA Extraction

Synchronized embryos or L4 larval worms were washed several times with M9 media to avoid contamination from bacteria, snap-frozen in liquid nitrogen and then ground to powder by a liquid nitrogen pre-chilled mortar and pestle. High-molecular weight and low-molecular weight RNA fractions were isolated using miRVana miRNA isolation kit (Ambion). RNA quantity was measured by Qubit® Fluorometer using Qubit® RNA HS Assay Kit (Molecular probes) and its quality was estimated by agarose gel electrophoresis and Tapestastion (Agilent genomics). First Choice Human Brain Total RNA, (Life Technologies) was used as the human brain RNA sample.

### Determining SPRI Binding Conditions

Volumes of PEG solution and Isopropanol were calculated using the equation:

X+(5PV/100)+(QV/100)=V

Where: V is total volume; X is volume of nucleic acid solution; P is desired concentration (%) of PEG; Q is desired concentration (%) of Isopropanol. First, a total volume of binding solution (V) was calculated by substituting P, Q and X in the equation for the desired concentrations of PEG and Isopropanol and the volume of nucleic acid solution. Next, the volumes of 20% PEG and 100% Isopropanol needed for the desired concentrations, being equal to 5PV/100 and QV/100, respectively, were calculated. To measure binding efficiency a solution of 2 ng/ul of synthetic single-stranded DNA oligonucleotide was aliquoted 50 ul per tube. To each tube SPRI beads in 20% PEG (SPRlselect, Beckman-Coulter) and 100% Isopropanol were added at volumes determined using the calculation method above. Size-selection was performed according to the manufacturer's protocol (Beckman's AMpureXP, left-side selection). Oligonucleotide concentrations in the input and eluted samples were measured by Qubit® Fluorometer using Qubit® ssDNA Assay Kit (Molecular probes). Binding efficiency was calculated by the percentage of the output oligonucleotide from the input quantity.

### Small RNA Library Preparation

Small RNA libraries were prepared from at least 3 biological replicas of N2 worms at embryo or L4 stage. One RNA sample from each stage was selected for preparing two additional libraries, resulting in 3 technical replicas for each stage. In short, sRNA was separated from other RNA species and then ligated to 5′-adenylated 3′-adapters using T4 RNA ligase-2 truncated (NEB) in an absence of ATP. The 3′-adapter-ligated sRNA was separated from free 3′-adapters and then ligated to a 5′-adapter, containing multiplexing barcode and UMI, using T4 RNA ligase 1 (NEB). sRNA ligated from both sides was then separated from the adapter-dimer to obtain an sRNA library. All the separation steps of the process of library preparation were performed using the method described herein. sRNA library was reverse-transcribed using QScript Flex cDNA synthesis kit (Quanta) and amplified using Phusion High-Fidelity DNA Polymerase (NEB). The amplified library was cleaned from primers and irrelevant products below 100 bp and above 200 bp by double-side size-selection on SPRI beads (Beckman's AMpureXP) and its concentration and quality was determined by Tapestation analysis (Agilent genomics). Libraries were sequenced using 50 basepair SR sequencing mode on HiSeq 2500 platform (Illumina)

### Sequence Processing and Expression Analysis

RNA sequences obtained were first de-multiplexed according to the 4-nucleotide barcode. Next, the 3′ adapter sequences were trimmed off by scanning from the 3′-end of the sequence at the first instance of the adapter sequence by increments of 1 nucleotide. Then either 1) removing the barcode and UMI (8-nucleotide) and these sequences are considered as non-collapsed or 2) merging identical sequences and then removed the barcode and UMI and these sequences are considered as collapsed.

C. elegans sequences were either aligned to the WS220 (Wormbase, www.wormbase.org) genome using Bowtie for size distribution analysis, allowing no mismatches with no more than 10 alignments to the genome or aligned to miRBase WBce1235 (www.mirbase.org), allowing no mismatches and not more than one alignment. Human brain sequences were aligned to miRBase GRCh38 with the same parameters.

Size distribution analysis was done on processed sequences before and after alignment to the genome. DEseq package in R (http://www.r-project.org) was used to evaluate miRNA expression and the Dispersions function in DEseq was used to estimate the dispersion between biological replicas and technical replicas.

### Example 1

### A Novel Method for Separating Nucleic Acids Shorter Than 100 Nucleotides

Most of the difficulties in sequencing and quantifying sRNAs derive from their small size and repetitive nature. To single them out and separate them from rRNA and tRNA and to separate ligation products from adapter-dimers, a technique pure and simple separation based on size of fragments ranging between 20 to 100 nucleotides (nt) is required. To achieve this, the SPRI bead isolation method was modified. This method makes use of size-selection based on a non-specific reversible binding of nucleic acid molecules to carboxyl groups coated magnetic beads in the presence of a “crowding agent” such as polyethylene glycol (PEG) and salt. As the efficiency of the binding is dependent on the length of the fragment and the concentration of the crowding agent, it is possible to separate two fragments of different lengths. It was hypothesized that by adding isopropanol (a second crowding agent) and adjusting its concentration in addition to PEG, it would be possible to achieve separation of molecules significantly shorter than 100 nt. Therefore, a series of SPRI-based size-selection solutions were prepared, all having the same concentration of PEG (7.5%), the same salt concentration (0.93M NaCl), but different concentrations of isopropanol, ranging from 32% to 54.5%, and their ability to promote binding of synthetic single stranded DNA oligonucleotides of different lengths to the beads was tested (see Materials and Methods). The oligonucleotides sizes, ranging from 19 to 66 nt, were chosen to cover the separation steps needed for sRNA library preparation, namely separation of 3′adapter ligated sRNA (34-45 nt) from free 3′adapter (18 nt), and separation of 3′ and 5′-adapter ligated sRNA (64-72 nt) from adapter dimer (45 nt). Binding efficiency was calculated by the ratio of oligonucleotide quantities in the eluent versus the input using a fluorimeter. The results of the experiment are summarized in Table 1.

TABLE 1
Binding (% of input) of sRNA molecules to beads at constant
PEG (7.5%) with varying isopropanol concentrations.

| Oligo | Isopropanol concentration (%) | | | | | | | | |
|---|---|---|---|---|---|---|---|---|---|
| size (nt) | 30 | 32 | 35 | 38 | 41 | 44 | 48 | 51 | 54.5 |
| 66 | 21 | 56 | 74 | 87 | 97 | 100 | ND | ND | ND |
| 58 | ND | 4 | 8 | 42 | 80 | 89 | ND | ND | ND |
| 44 | ND | 2 | 3 | 13 | 40 | 59 | 75 | ND | ND |
| 37 | ND | ND | 1.5 | 4 | 11 | 19 | 45 | 80 | ND |
| 30 | ND | ND | ND | ND | 4 | 5 | 20 | 40 | 61 |
| 21 | ND | ND | ND | ND | ND | 2 | 4 | 7 | 15 |
| 19 | ND | ND | ND | ND | ND | 1 | 3 | 3 | 5 |

ND = not determined.

As hypothesized, increasing concentration of isopropanol led to increased binding efficiency of smaller oligonucleotides. Moreover, for each oligo length tested, an isopropanol concentration that resulted in significant binding (>40%) to the beads, coupled with oligos ˜20 nucleotides shorter being poorly bound (<5%) was found. It is to be understood that if 100% of oligos with a specific length bound the beads at a given concentration of isopropanol, then increasing the concentration would also result in 100% binding, however it would also result in increased binding of smaller oligos.

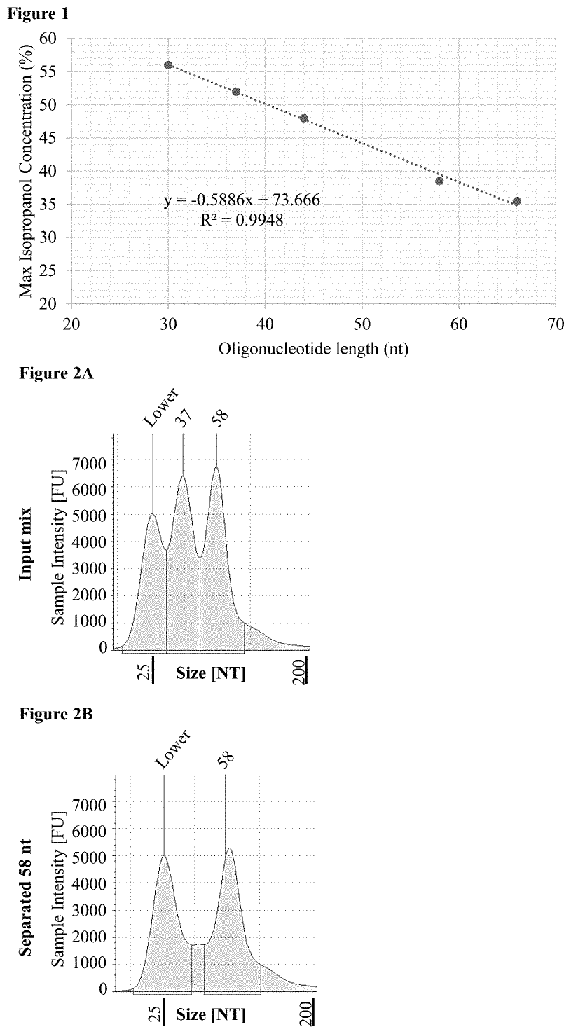

These experimentally arrived upon ideal conditions are summarized in Table 2. Based on these experiments a theoretical range of concentrations that would result in significant binding coupled with ˜20 nucleotides shorter oligos binding poorly could be hypothesized (Table 2). Further a hypothetical isopropanol concentration for the highest yield of oligos of a specific length, with less than 5% contamination of oligos ˜20 nucleotides shorter, can be easily arrived upon. With these ideal values in hand the six tested oligo lengths (66, 58, 44, 37, 30 nt) were plotted against their ideal isopropanol concentrations (FIG. 1). A best fit line was calculated and found to have a slope of −0.589. In other words, for every decrease in length of one nucleotide, one should perform the isolation with a concentration of isopropanol increased by 0.589%. If one sets the isopropanol concentration for isolating oligonucleotides of length 58 at 38.5% (this size has the smallest acceptable range of those tested), then a theoretical isopropanol concentration for extraction of an oligo nucleotide of any length from 99 to 10 can be calculated. Further, as evidenced by the range of values that were effective, 4% above or below this theoretical value may be effective for isolation. For technical reasons isopropanol concentration could not be raised about 54.5%, as this led to aggregation of the beads and poor nucleic acid binding.

TABLE 2
Experimental and hypothetical ideal isopropanol
concentrations for nucleotide isolation.

| Oligo size | Experimental ideal iso. | Hypothetical ideal | Iso. conc. for |
|---|---|---|---|
| (nt) | conc. (%) | iso. conc. range | maximum yield |
| 66 | 32 or 35 | 31-35.5 | 35.5 |
| 58 | 38 | 38-38.5 | 38.5 |
| 44 | 41, 44 or 48 | 41-48 | 48 |
| 37 | 48 or 51 | 47.5-52 | 52 |
| 30 | 51 or 54.5 | 51-54.5 | 54.5 |

### Example 2

### Increased PEG Concentration Also Improves Binding of Smaller Nucleic Acid Molecules

It was next tested if increasing the PEG concentration could increase the binding of extremely small nucleic acid molecules. Oligonucleotides 21 bases long were extracted using 54.5% ethanol and 0.93M NaCl but with two different concentrations of PEG. At a PEG concentration of 7.5% (used in above described experiments) 13-15% of the oligo could be bound to beads. When the PEG concentration was increased to 8.0%, the yield increased to 18-21% of the input.

### Example 3

### Separating 58 and 37 nt Long Oligonucleotides

The feasibility of using these conditions to separate two oligonucleotides that differed in length by 21 nt (37 nt and 58 nt) was tested. Double-sided size selection on the SPRI beads was used, which entailed 1) binding the longer fragment to the beads and collecting the unbound material containing the shorter fragments (right-side size-selection), and 2) adding a second batch of beads, and adjusting the conditions (based on Table 1) to allow complete binding of the shorter fragments (left-side size selection). Eluting the beads from the right-size selection will isolate the longer fragments and eluting the beads from the left size selection will provide the shorter fragments. For isolating the 58 nt oligos, three different concentrations of isopropanol for the right-side size selection, 38%, 41%, and 44% were used. The supernatant, containing the unbound shorter oligonucleotide, was transferred to a new tube and a left-size selection was performed with 54.5% isopropanol to allow maximal recovery of the 37 nt oligos.

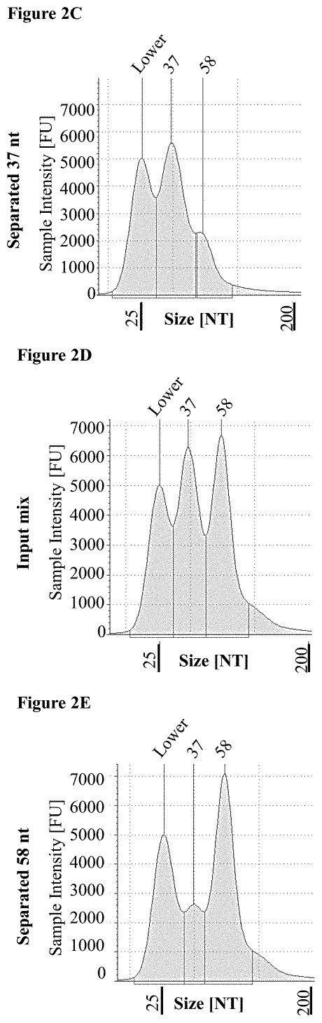

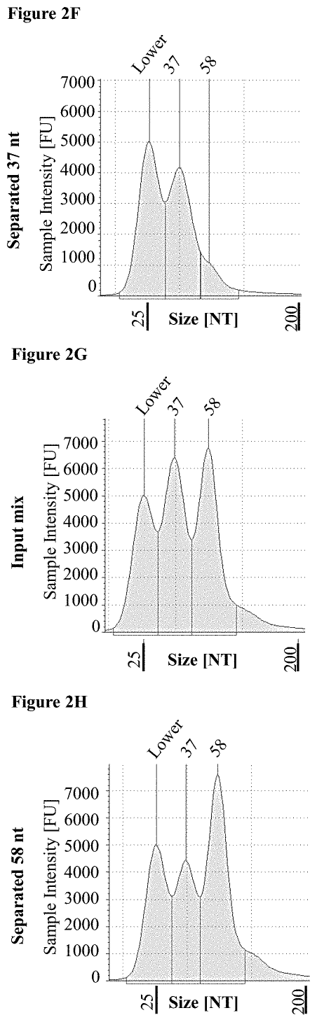

The input mixture and eluates from each size-selection step were analyzed using Tapstation, with a 25 nt size marker added to every run (FIG. 2A-I). Binding efficiencies were consistent with those determined using a single size of oligo (Table 1). Using 38% Isopropanol for the right-size selection recovered around two-thirds of the 58 nt input material with minor left overs of the 37 nt oligos (FIG. 2A-B), while the left-size selection resulted in nearly a complete recovery of the 37 oligos, but a third of the input material of the 58 nt oligos (FIG. 2C). A mirror picture was obtained using 44% Isopropanol, right-size selection resulted in a complete recovery of the 58 nt oligos with a noticeable fraction of 37 nt oligos (FIG. 2G-H), while the left-size size selection yielded a third of the 37 nt input oligos with almost no 58 nt oligos (FIG. 2I). In between results were observed when using 41% Isopropanol (FIG. 2D-F). Thus, using the concentrations of isopropanol presented in Tables 1 and 2, it is possible to separate between two short nucleic acids differing by ˜20 nt with high recovery.

### Example 4

### Separating Ligation Products From Ligation Byproducts

By using different concentration of PEG and isopropanol as described in Tables 1 and 2, the inventors were able to differentiate between molecules of small sizes.

For example, in order to separate ligation products (also termed herein, “outcome of a ligation procedure”) comprising sRNAs ligated to an oligonucleotide (40b) from the unligated input molecules such as free oligonucleotide (18b) and small RNA (22b), a binding solution comprising 7.5% PEG, 0.9 M Nacl and 48% isopropanol was used. The binding solution was formed by adding 130 microliter (μl) of Ampure beads (in 20% PEG, 2.5 M NaCl) and 165 μl of 100% isopropanol to 50 μl of a ligation product. According to table 1, under these condition ˜75% of the small-RNA ligated to an oligonucleotide (40 nt) should be bound to the bead, and only up to 5% of the byproducts should be bound.

In order to separate ligation products comprising sRNAs ligated to oligonucleotides at both ends (67 nt) and ligation byproducts such as oligonucleotide dimers (45 nt), unligated input molecules (sRNAs and one oligonucleotide, 40b), and free 5′ -oligonucleotide (27b), a solution comprising 7.5% PEG, 0.9 M Nacl and 35% isopropanol was used. The binding solution was formed by adding 69 μl of Ampure beads (20% PEG, 2.5 M NaCl) and 64 μl of 100% Isopropanol to 50 μl of a ligation product. According to table 1, under these condition around 74% of the small-RNA ligated to oligonucleotides at each end should be bound to the particle, and less than 4% of the byproducts should be bound.

These examples demonstrate the manner in which the methods of the instant invention can be used in order to purify the products of a ligation procedure from the byproducts and input of the ligation procedure.

### Example 5

### QsRNA-seq—A Method for Preparation of Small RNA Libraries

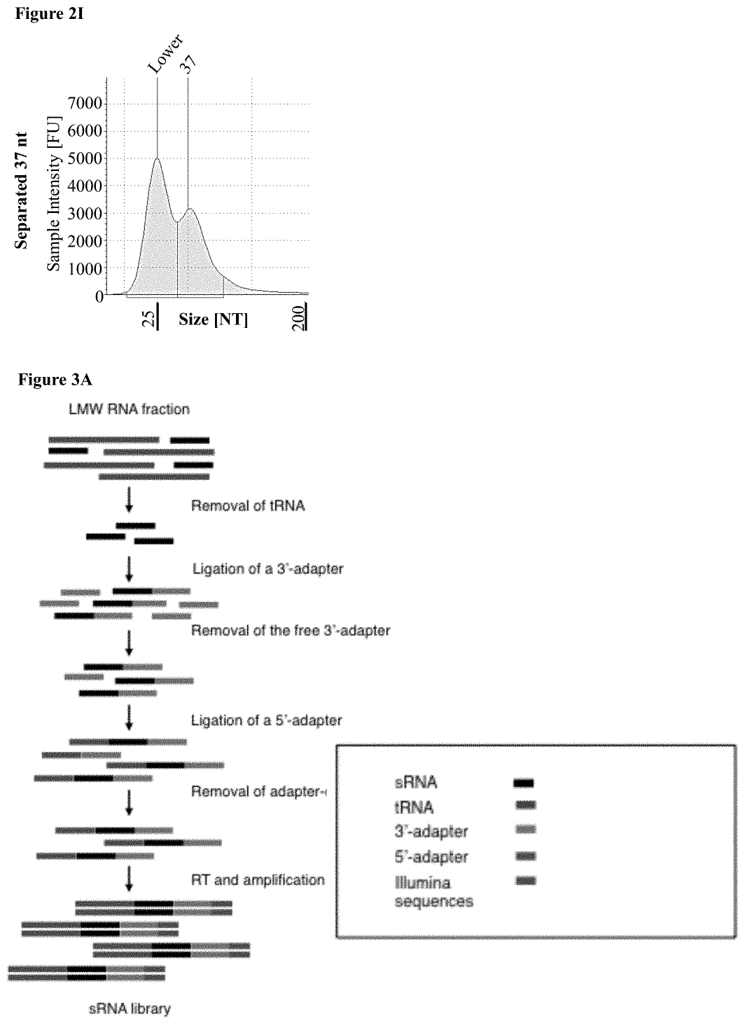

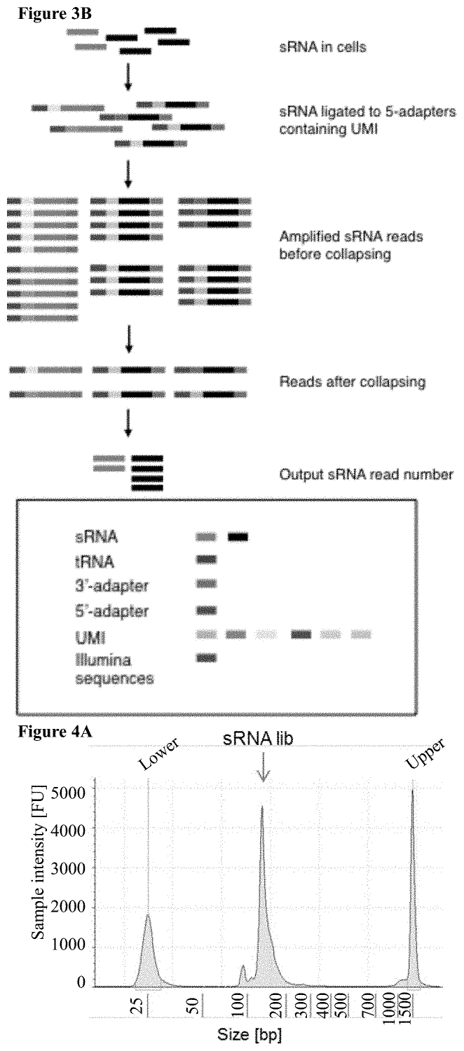

The above described separations were incorporated into a new protocol for preparation of small RNA libraries for high-throughput sequencing. The protocol, named QsRNA-seq, is presented in FIG. 3A (for detailed protocol see Materials and Methods). The protocol implements two ligation steps: 1) ligation of pre-adenylated 3′-adapter without ATP and 2) ligation of 5′-adapter containing a 4-nt barcode to allow multiplexing. Once only RNA of less than 100 bp in length is obtained, three size separation steps on SPRI magnetic beads are performed to obtain only the required RNA molecules: 1) separation of very small RNA (<40 nt) from longer RNAs (being mainly tRNAs) prior to the first ligation; 2) separation of 3′-adapter ligated small RNA from free 3′- adapter following the first ligation; and 3) separation of 3′,5′-adapter ligated small RNA from adapter-dimer and free 5′-adapter following the second ligation (sizes of fragments with and without a UMI barcode for multiplexing are provided in Table 3.

TABLE 3
Sizes of nucleic acid fragments used and generated during
QsRNA-Seq library preparation. Numbers in parenthesis
correspond to expected sizes of a miRNA-based library.

| | No UMI | 8nt-long UMI | |
|---|---|---|---|
| | barcode (0N) | barcode (8N) | |
| Small RNA | 19-27(22) | 19-27 | (22) |
| 3′-adapter | 18 | 18 | |
| 5′-adapter | 19 | 27 | |
| 3′-ligated small RNA | 37-45 (40) | 37-45 | (40) |
| 3′,5′-ligated small RNA | 56-64 (59) | 64-72 | (67) |
| Adapter-dimer | 37 | 45 | |
| Amplification products: | | | |
| Final library | 116-124 (119) | 124-132 | (127) |

| | | |
|---|---|---|
| Adapter-dimer | 97 | 105 |

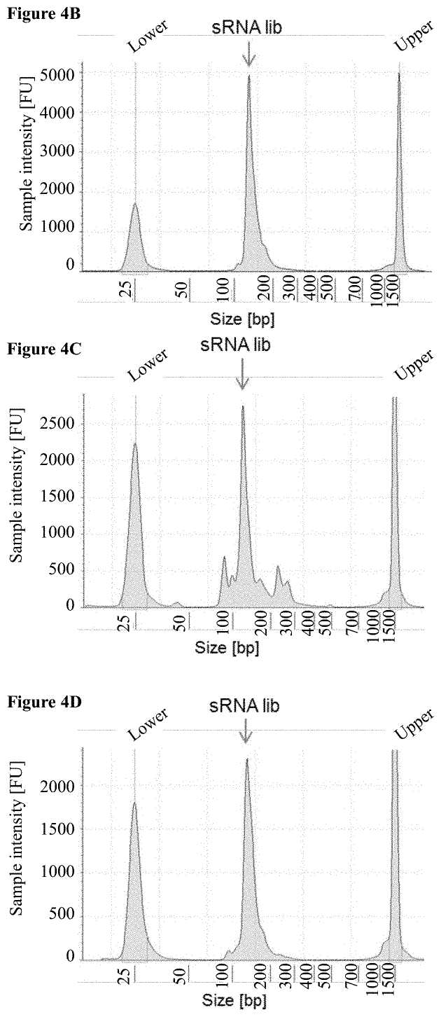

In order to correct for PCR-induced artifacts and enable quantification 5′ adapters that contain 8 random nucleotides that provide a unique identifier to each RNA molecule (UMI) were used. After PCR amplification, identical small RNAs with the same UMI were considered an amplification product and merged to one sequence (i.e., collapsing, FIG. 2B). To test the ability of QsRNA-seq to detect sRNAs, QsRNA-seq was performed on RNA extracted from wildtype C. elegans synchronized to embryo or L4 larval stage and on total RNA obtained from human brain tissue. Human brain total RNA was chosen because miRNAs constitute most of the small RNAs in this sample, thus a very uniform library was expected. In contrast, C. elegans contains many types of small RNA, including miRNAs, primary and secondary endogenous siRNAs, and piRNAs. 3 independent biological samples from each C. elegans developmental stage were generated as biological replicates. RNA extracted from one sample from each stage was also subjected to 3 independent library preparations, as technical replicates, and was also used to prepare 3 replicate libraries having no UMI in the 5′-adapter (ON). All library preparations resulted in very clean products after PCR amplification (FIG. 4A, 4C) which were ready for sequencing with negligible amounts of adapter-dimers (less than 2% of total reads in each library). To achieve even greater purity, a SPRI size selection with PEG alone could also be performed (FIG. 4B, 4D).

### Example 5

### QsRNA-seq Can Evaluate miRNAs Abundance and Expression Changes Accurately

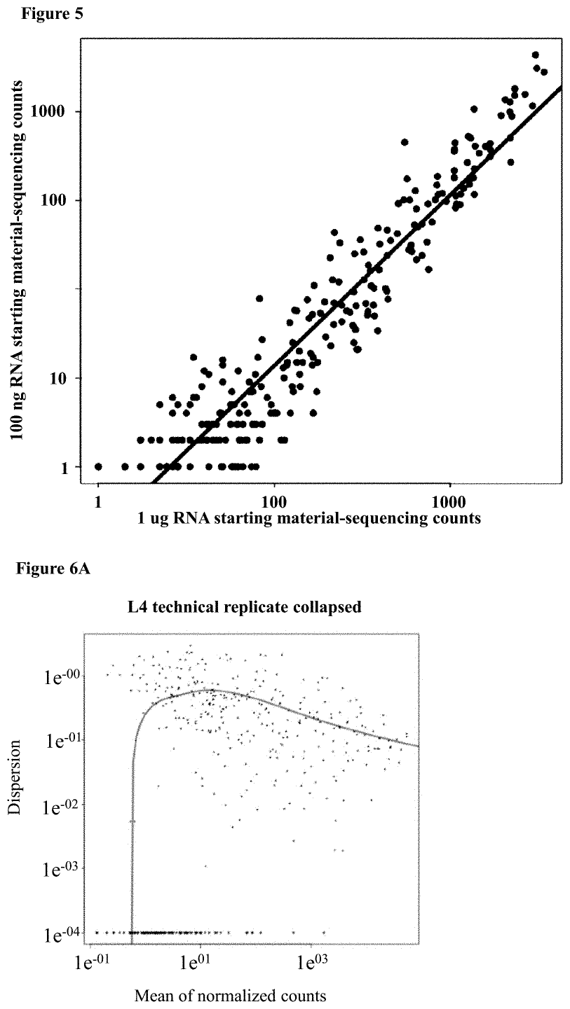

To evaluate the quality of the QsRNA-seq output sequences, the generated sequences were aligned to all annotated miRNAs (both miRNA and miRNA*) in the C. elegans, miRbase WBce1235, or in the human, miRBase GRCh38. In the C. elegans samples, all the annotated miRNAs were present in QsRNA-seq samples by at least one strand (3P or 5P), while 97% of all microRNAs, had coverage for both strands. Even rare miRNAs such as lys-6, which is expressed in only one pair of neurons in the C. elegans head were present. QsRNA-seq allows for extensive multiplexing of the samples before amplification, which can reduce significantly the amount of starting material required; however, even without multiplexing, reducing the starting material by 10-fold, from lug to 100 ng, produced nearly identical results (FIG. 5). In human brain, the coverage was somewhat lower, with alignment to 80% of annotated miRNAs. The difference likely derives from the pooled large number of samples that were generated from whole worms at two developmental stages while only one sample from human cells was generated. However, miRNAs known to be enriched in human brain, for example, the let-7 family, mir-9, mir-26a and others were very abundant in the brain libraries.

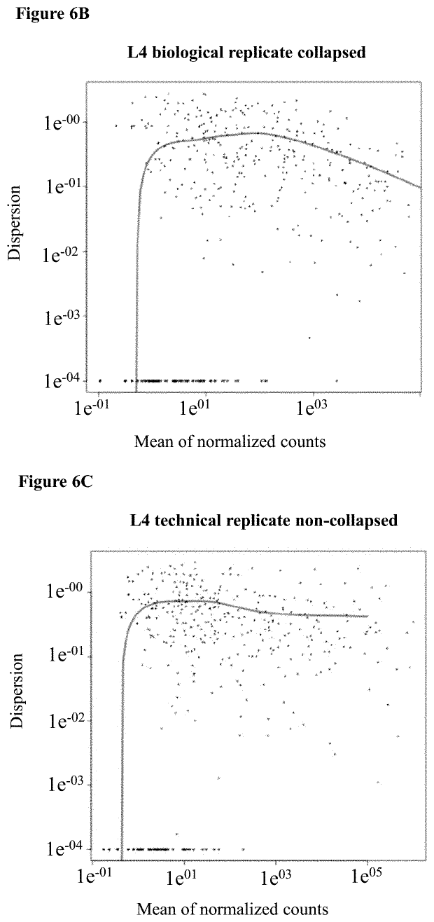

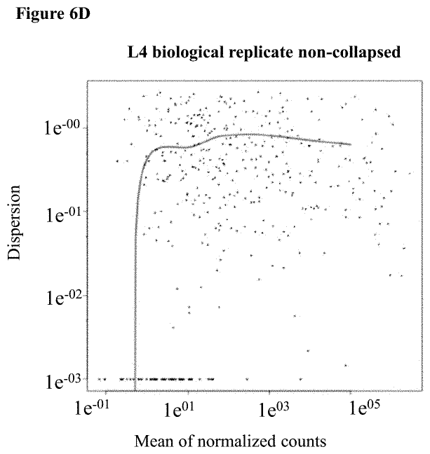

To assess the consistency of the method, the dispersion of miRNA expression between the replicates, biological and technical, collapsed and non-collapsed was evaluated. As expected, the collapsed replicates exhibited lower dispersion rates than the corresponding non-collapsed replicates, for both biological and technical replica types (FIG. 6A-D). For example, comparing the dispersion of the biological samples at embryo stage L4 between collapsed reads (FIG. 6A-B) and non-collapsed reads (FIG. 6C-D), at high normalized counts (>e+03) it was observed that the dispersion in the collapsed samples ranged between about e-0.5 and e-0.8, whereas the non-collapsed counts ranged between e-0.125 and e-0.22. Moreover, collapsed count dispersion decreases as the mean of the normalized counts increases, thus confirming the assumption that collapsing will tend to reduce statistical errors more drastically when dealing with larger counts.

### Materials and Methods

Ligation of Adenylated 3′-oligonucleotide to 3′ End of Small RNA

Denaturation of the secondary structure of the small RNA and the oligonucleotide was performed by incubation at 72° C. for −3 min Next, ligation of an oligonucleotide to 3′ end of small RNA molecules was performed by incubating the denaturated small RNA molecules and the oligonucleotides together with RNAse inhibitor, T4RNA ligase 2 truncated, T4 ligase buffer at 25° C. for 1 h. The total reaction volume was 30 μl (Table 4). The oligonucleotide was IDT-Linker-1, which is 18b long, 5′-adenylated and 3′ blocked.

TABLE 4
Reagents for ligation reaction of 5′ adenylated
3′-oligonucleotide to small RNA

| | | |
|---|---|---|
| Small RNA | 18.75 | μl |
| cloning Linker-1 (100 uM) | 0.5 | μl |
| RNAse inhibitor (40 u/ul) | 0.75 | μl |
| 10X T4 ligase buffer | 3.0 | μl |
| 50% PEG 8000 | 6.0 | μl |
| T4 RNA ligase 2 truncated | 1.0 | μl |
| Total | 30 | μl |

Ligation of 5′-oligonucleotide to 5′ end of 3′ Ligated Small RNA

Denaturation of the secondary structure of the 3′ ligated small RNA molecules and oligo reverse-complementary to 3′-oligonucleotide (such as RT primer) was performed by incubation at 72° C. for 3 min followed by annealing at 37° C. for 5 min to allow annealing of reverse-complementary oligo to free 3′-olinucleotide (if present), to prevent formation of oligonucleotide dimer during ligation. The denaturation of the secondary structure of 5′-oligonucleotide was performed by incubation at 72° C. for 3 min in separate tube. Next, ligation of 5′-oligonucleotide to 3′ ligated small RNA molecules was performed by incubating the denaturated pre-annealed small RNA molecules and the 5′-oligonucleotide together with RNAse inhibitor, T4RNA ligase 1, T4 ligase buffer, ATP at 37° C. for lh. The total reaction volume was 30 μl (Table 5). A 27b long 5′-oligonucleotide was used. The 5′-oligonucleotide comprised a 12b barcode which comprised a sequence of 8 random nucleotides.

TABLE 5
Reagents for ligation reaction of 5′-oligonucleotide
to 3′ ligated small RNA

| 3′ ligated small RNA | 19.00 | ul |
|---|---|---|
| blocking oligo (100 μM) | 1.0 | ul |
| Barcoded 5′ oligonucleotide (100 μM) | 0.5 | ul |
| RNAse inhibitor (40 μ/μl) | 1 | ul |
| T4 RNA Ligase Buffer (NEB) | 3 | ul |
| ATP (10 mM) | 3 | ul |
| T4 RNA ligase1 | 1 | ul |
| H2O | 1.5 | |
| TOTAL | 30 | ul |

The blocking oligo is used for preventing formation of oligonucleotide -dimer by ligation of free 3′-oligonucleotide, if some of it remained unremoved by previous step, to 5′-oligonucleotide

### Reverse Transcription of 3′ and 5′ Oligonucleotide Ligated Small RNA

Denatration of the secondary structure of the 3′ and 5′ ligated small RNA molecules was performed by incubation of 12.5 ul of 3′ and 5′ ligated small RNA together with 0.5 ul of 100 μM RT Primer, and 2 ul of GSP enchancer at 65° C. for 5 min Next, −4 ul of RT reaction mix and lul of qScript reverse transcriptase (Quanta Biosciences) were added, and the mixture was incubated at 42° C. for 60 minutes. Next, RT enzyme inactivation was achieved by incubating the mixture at 85° C. for 5 min The resulting cDNA were than amplified.

cDNA Amplification

For cDNA amplification, a total reaction volume of 50 μl comprising 4 μl RT product, 33 μl H2O, 0.5 μl FWD primer (100 μM), 0.5 μl REV primer (100 μM), 10 μl of 5× buffer, 1 μl of dNTPs (10 mM), 1 μl of Phusion polymerase was used. PCR reaction was performed as follows: heating to 98° C. for 30 seconds; 14 to 26 cycles of 98° C. for 10 seconds; 50° C. for 30 seconds; and 72° C. for 15 seconds; followed by a final extension at 72° C. for 2 minutes.

### Preparation of Small RNA Libraries

The methods of the instant invention can be used for the preparation of small RNA libraries. In an exemplary protocol described herein, the methods for isolating or excluding small RNA molecules of the instant invention are used in order to isolate the desired products of enzymatic procedures such as ligation, reverse transcription etc. In the exemplary protocol Ampure SPRI beads (Beckman coulter) are used. In addition, the small RNA is ligated to an oligonucleotide comprising a barcode comprising a random sequence in order to distinguish between original small RNA molecules and amplified copies thereof. In the exemplary protocol, a low molecular weight (lmw) RNA fraction obtained using Mirvana kit (Ambyon) is used as a starting material for small RNA library preparation. However, total RNA extracted by other kit/method preserving small RNA can be used as well. In such a case RNA having a length of more than 100 bases (b) can be removed using Ampure SPRI beads (Beckman coulter) and standard manufacturer's protocol.

First, small RNA molecules (20-30b) are separated from longer RNA molecules (40b-100b, including tRNA (about 60 bases). Isolation of small RNA is achieved by using two separation steps. In the first separation step beads and isopropanol are added to RNA to obtain a binding solution comprising conditions (Table 6) for binding RNA molecules of at least 40 bases to the beads. Therefore, following the first separation step, small RNA molecules are obtained in the supernatant. In the second separation step beads and Isopropanol are added to the supernatant to obtain a binding solution comprising conditions (Table 7) for binding RNA molecules of at least 20 bases is used. Therefore, following the second separation step, small RNA molecules are bound to the beads.

TABLE 6
Conditions for the first separation step

| | Ampure BEADS (in | | Isopro- | | Total |
|---|---|---|---|---|---|
| Sample | 20% PEG 2.5M NaCl) | Peg % | panol | Isop % | Volume |
| 50 ul | 100 ul | 7.5% | 115 ul | 44% | 265 ul |

TABLE 7
Conditions for the second separation step

| | | | Isopro- | | Total |
|---|---|---|---|---|---|
| sample | AMPure beads | Peg % | panol | Isoprop % | volume |
| 265 ul | 150 ul for total of 250 | 7.5% | 245 ul | 54.5% | 660 ul |

These values are due to the sample already contains 100 μl PEG and 115 μl isopropanol from the previous step. Increasing the PEG concentration to 7.78% was also found to be effective in isolating the desired RNA molecules.

The small RNA molecules obtained by the previous step are then ligated to oligonucleotides. Following the ligation of oligonucleotides to the 3′ end of the small RNA molecules (see above) the 3′ ligated small RNA molecules (38-48b) are separated from the ligation byproducts consisting of oligonucleotides (18b) and small RNA molecules (20-30b), the separation is performed under suitable conditions for the separation (Table 8).

TABLE 8
Conditions for a separation of small RNA molecules
having a length of at least 37 bases from small
RNA molecules having a length of at most 22 bases

| | | | Isopro- | | Total |
|---|---|---|---|---|---|
| Sample | Ampure beads | Peg % | panol | Isop % | volume |
| 65 ul | 115 ul | 7.5% | 165 ul | 48% | 345 ul |

The ligation reaction already contains 6 μl of 50% PEG 8000 which equals to 15 μl of PEG 20%; to obtain the desired separation conditions additional 15 μl H2O are added to the sample (obtained sample equals to 50 μl RNA in water +15 μl 20% PEG8000; respectively the amount of PEG solution added is reduced by 15 μl.

The 3′ ligated small RNA obtained by the previous step are than ligated to 5′-oligonucleotide comprising a barcode. Following the ligation of oligonucleotides to the 5′ end of the 3′ ligated small RNA molecules, the small RNA molecules ligated to two oligonucleotides (65-75b) are separated from the ligation byproducts such as 5′-oligonucleotides (27b), 3′ ligated small RNA molecules (38b-48b) and a dimer of 3′-5′ oligonucleotides (45b), the separation is performed under suitable conditions for the separation (Table 9).

TABLE 9
Conditions for a separation of small RNA molecules
having a length of at least 64 bases from small
RNA molecules having a length of at most 45 bases

| | Ampure beads | | Isopro- | | Total |
|---|---|---|---|---|---|
| Sample | (in 20% PEG) | Peg % | panol | Isop % | volume |
| 50 ul | 69 ul | 7.5% | 64 ul | 35% | 170 ul |

Next, reverse transcription is performed on the small RNA molecules ligated at their 3′ and 5′ ends. Lastly, the product of reverse transcription is amplified by using a PCR.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-48. (canceled)

49. A method for separating a nucleic acid molecule of a desired length below 100 nucleotides from a solution comprising a nucleic acid molecule of the desired length and a nucleic acid molecule of a length at least 15 nucleotides shorter, the method comprising:
(a) obtaining a solution comprising nucleic acid molecules of multiple lengths;
(b) adding to said solution particles comprising a carboxyl-group coated surface, salt, polyalkylene glycol, and alcohol; and
(c) isolating said particles;
thereby separating a nucleic acid molecule of a desired length below 100 nucleotides from a solution comprising a nucleic acid molecule of the desired length and a nucleic acid molecule of a length at least 15 nucleotides shorter.

50. The method of claim 49, wherein said salt is sodium chloride, said polyalkylene glycol is polyethylene glycol (PEG), said alcohol is isopropanol and wherein said adding produces a binding solution having concentrations of PEG and isopropanol suitable for selective binding of said nucleic acid molecule of a desired length to said particles; wherein
(i) said desired length is at least 60 bases and said concentration of PEG and isopropanol is 7%-8.5% and 32%-41%, respectively;
(ii) said desired length is at least 50 bases and said concentration of PEG and isopropanol is 7%-8.5% and 38%-45%, respectively;
(iii) said desired length is at least 40 bases and said concentration of PEG and isopropanol is 7%-8.5% and 41%-50%, respectively; and
(iv) said desired length is at least 30 bases and said concentration of PEG and isopropanol is 7%-8.5% and 45%-58%, respectively; and
(v) said desired length is at least 20 bases and said concentration of PEG and isopropanol is 7%-8.5% and 49%-60%, respectively.

51. The method of claim 49, wherein
i. said polyalkylene glycol is polyethylene glycol (PEG);
ii. said alcohol is isopropanol;
iii. said salt is sodium chloride (NaCl); or
iv. a combination thereof.

52. The method of claim 49, wherein
i. said final concentration of salt is between 0.8 and 1 molar;
ii. said final concentration of polyalkylene glycol is between 7.0% and 8.5%, or optionally between 7.5% and 8.0%.; or
iii. said final concentration of alcohol is about 73.7% minus 0.59% times said desired length in nucleotides.

53. The method of claim 49, wherein said final concentration of alcohol is between 67% minus 0.59% times said desired length in nucleotides and 75% minus 0.59% times said desired length in nucleotides, optionally wherein said final concentration of alcohol is between 70% minus 0.59% times said desired length in nucleotides and 74% minus 0.59% times said desired length in nucleotides.

54. The method of claim 49, further comprising incubating the solution of step (b) for an amount of time sufficient for binding of the desired nucleic acid molecule to said particles prior to step (c).

55. The method of claim 49, wherein said separating results in less than a 10% contamination by said nucleic acid molecule 15 nucleotides shorter than the desired length.

56. The method of claim 49, wherein said particles are separated or isolated by a method selected from the group of methods consisting of: applying vacuum filtration, magnetic separation and centrifugation.

57. The method of claim 49, wherein said nucleic acid molecule of a desired length is one of the following: a single-stranded nucleic acid molecule, a double-stranded nucleic acid molecule, a small RNA and a ligation product.

58. The method of claim 49, wherein said solution comprising nucleic acid molecules is selected from: an outcome of a reverse transcription procedure, extracted cellular RNA, a cell lysate, an outcome of an amplification procedure, an outcome of a ligation procedure, and an outcome of a restriction enzyme digestion.

59. The method of claim 57, wherein said ligation procedure comprises ligating a nucleic acid molecule to at least one of the following: a first oligonucleotide at said nucleic acid molecule's 3′ end, a second oligonucleotide at said nucleic acid molecule's 5′ end, and a first oligonucleotide at said nucleic acid molecule's 3′ end and a second oligonucleotide at said nucleic acid molecule's 5′ end.

60. The method of claim 58, wherein at least one of said first and second oligonucleotides comprise at least one of:
i. a nucleotide barcode; and
ii. a random sequence; optionally wherein said random sequence uniquely identifies said nucleic acid molecule and distinguishes between an original nucleic acid molecule and amplified copies thereof.

61. The method of claim 49, further comprising at least one of:
i. discarding supernatant from said reaction vessel;
ii. washing said particles; and
iii. eluting said nucleic acid molecule of a desired length from said particles by applying an aqueous solution.

62. A method for preparing a small RNA library, the method comprising:
(a) obtaining a first solution comprising RNA molecules shorter than 100 nucleotides and substantially depleted of RNA molecules longer than 100 nucleotides;
(b) removing from said first solution RNA longer than 40 nucleotides by adding to said first solution particles comprising a carboxyl-group coated surface, salt to a final concentration of between 0.8 and 1 molar, polyalkylene glycol to a final concentration of between 7 and 8.5%, and alcohol to a final concentration of between 41 and 49% and subsequently removing said particles;
(c) isolating from said first solution RNA longer than 19 nucleotides by adding to said first solution particles comprising a carboxyl-group coated surface, salt to a final concentration of between 0.8 and 1 molar, polyalkylene glycol to a final concentration of between 7 and 8.5%, and alcohol to a final concentration of between 53 and 60.0% and subsequently isolating said particles and optionally eluting said RNA longer than 19 nucleotides into a second solution,
(d) ligating a 3′ adapter to said isolated RNA longer than 19 nucleotides;
(e) isolating RNA ligated to a 3′ adapter by adding particles comprising a carboxyl-group coated surface, salt to a final concentration of between 0.8 and 1 molar, polyalkylene glycol to a final concentration of between 7 and 8.5%, and alcohol to a final concentration of between 45 and 52% and subsequently isolating said particles and optionally eluting said RNA ligated to a 3′ adapter into a third solution;
(f) ligating a 5′ adapter to said isolated RNA ligated to a 3′ adapter;
(g) isolating RNA ligated to a 3′ and 5′ adapter by adding particles comprising a carboxyl-group coated surface, salt to a final concentration of between 0.8 and 1 molar, polyalkylene glycol to a final concentration of between 7 and 8.5%, and alcohol to a final concentration of between 32 and 44% and subsequently isolating said particles;
thereby preparing a small RNA library.

63. The method of claim 60, wherein said solution of step (a) is depleted of RNA molecules longer than 100 nucleotides by use of a kit for extraction of high molecular weight nucleic acids.

64. The method of claim 60, wherein said 5′ adapter comprises a barcode or a random sequence and optionally wherein said random sequence uniquely identifies a RNA molecule and can distinguish between an RNA originally in the solution of step (a) and an amplified copy thereof.

65. The method of claim 60, wherein
i. the alcohol in step (b) is at a final concentration of about 44%;
ii. the alcohol in step (c) is at a final concentration of about 54.5%;
iii. said 3′ adapter is about 18 nucleotides long, and the alcohol in step (e) is at a final concentration of about 48%;
iv. said 5′ adapter is between 19 and 37 nucleotides long and the alcohol in step (g) is at a final concentration of between 35 and 38%;
v. said 5′ adapter is about 27 nucleotides long and the alcohol in step (g) is at a final concentration of about 35% or
vi. a combination thereof.

66. The method of claim 60, further comprising at least one of
i. adding a blocking oligo to said isolated RNA after step (e);
ii. eluting said isolated RNA longer than 56 nucleotides from said particles by applying an aqueous solution;
iii. reverse transcribing said isolated RNA longer than 56 nucleotides into cDNA and optionally PCR amplifying said cDNA; and
iv. washing said particles following every isolation.

67. A kit for isolating and separating nucleic acid molecules of a desired length below 100 nucleotides, the kit comprising:
(a) at least one of the following: (i) a table of efficiencies of binding of nucleic acid molecules of different lengths to particles comprising a carboxyl-group coated surface for a range of concentrations of PEG and isopropanol, and (b) an equation for calculating ideal isopropanol and PEG concentration for binding a nucleic acid molecule to a carboxyl-group coated surface and
(b) at least one of the following: (i) particles comprising carboxyl group coated surfaces; (ii) PEG; and (iii) isopropanol.

68. The kit of claim 67, for use in preparing a small RNA library, wherein the kit further comprises instruction for preparing a small RNA library and at least one of the following components: (i) 3′-oligonucleotides; (ii) 3′-oligonucleotides comprising an adenylated 5′ end; (iii) 5′-oligonucleotides; (iv) an oligonucleotide comprising a nucleotide barcode comprising a random sequence; (v) an RNA ligase; (vi) a reverse transcriptase; and (vii) a DNA polymerase.
